(19) United States
(12) Patent Application Publication
(10) Pub. No.: US 2006/0286545 A1
(43) Pub. Date: Dec. 21, 2006

(54) Viral vectors with improved properties

(75) Inventors: Thomas Weber, New York, NY (US); Laure Gigout, New York, NY (US)

Correspondence Address: DARBY & DARBY P.C., P. O. BOX 5257, NEW YORK, NY, 10150-5257, US

(73) Assignee: Mount Sinai School of Medicine of New York University, New York, NY (US)

(21) Appl. No.: 10/558,394

(22) PCT Filed: May 24, 2004
(86) PCT No.: PCT/US04/16382

§ 371 Date: Nov. 22, 2005

Related U.S. Application Data
(60) Provisional application No. 60/473,329, filed on May 23, 2003.

(51) Int. Cl. C40B 30/06; C40B 40/08; C12N 15/86

(52) U.S. Cl. 435/5; 435/6; 435/456; 435/325

(57) ABSTRACT

Methods to improve the tropism or other features of a virus is disclosed. Such methods can be used to prepare, e.g., DNA or plasmid libraries of variants of a gene encoding a viral capsid or envelope restriction site, libraries of viral clones with such variant genes with a randon-dy inserted restriction site or polypeptide sequence targeting a receptor expressed by a specific type of mammalian cells. Described are also methods to prepare mosaic viruses, i.e., viral particles wherein copies of one or more capsid or envelope proteins originate from different sources. These methods can be used to prepare mosaic viruses of a specific mixture of wild-type and mutant proteins, or of different types of mutant proteins.

This application claims priority under 35 U.S.C. § 119(e) from Provisional Application No. 60/473,329, filed May 23, 2003.

### FIELD OF THE INVENTION

This invention is in the field of viral vectors for use in gene therapy and other applications. The invention relates to methods and compositions for improving the ability of viral vectors to target and/or infect cells, as well as to plasmid and viral particle libraries encoding viral proteins.

### BACKGROUND OF THE INVENTION

Gene therapy has great promise for the treatment of a vast array of diseases; these include, but are by no way limited to, such important diseases as type I diabetes, degenerative brain disorders like Alzheimer and Parkinson, hematological diseases such as sickle cell anemia, other classical genetic disorders such as cystic fibrosis and lysosomal storage disorders and even diseases such as cardiovascular disorders and cancer. Despite its solid scientific rationale, however, examples of successful clinical applications of gene therapy remain scarce.

One of the main reasons for the, as of yet, limited success of gene therapy is the lack of ideal gene delivery vehicles. For example, most gene delivery vehicles applied clinically insert their DNA randomly into the genome of the patient. This was recently demonstrated to be a severe problem for therapeutic applications. In the only reported clinical success of gene therapy (Hacein-Bey-Abina et al., N Engl J Med, 2002; 346:1185-1193), the treatment of several children with Severe Combined Immuno Deficiency (SCID) recently suffered a severe setback. While the treatment was curative in four of the treated children, two of the children developed malignant disorders as a result of vector integration into oncogenic sites (Hacein-Bey-Abina et al., N Engl J Med, 2003; 348:255-256). Further, in experimental settings, transgene expression driven by expression cassettes that are integrated randomly into the genome of an experimental animal often declines over time. This reduction in expression can be dramatic even in the absence of an immune response. Perhaps the most important reason for this decline in transgene expression is the so-called “silencing” that can occur if the vector DNA is integrated into certain sites in the genome. These sites appear to occur with a frequency can be problematic for gene therapeutic application.

To date, most gene therapy clinical trials have been performed with viral vectors. The most common viral vectors used for these studies have been based on adenoviruses and retroviruses. Recently, another type of viral vectors, those based on adeno-associated virus (AAV), have emerged as promising candidates for gene therapeutic applications. The various serotypes of AAV are attractive for several reasons, most prominently that AAV is non-pathogenic and that the wildtype virus can integrate its genome site-specifically into human chromosome 19 (Linden et al., Proc Natl Acad Sci USA, 1996; 93:11288-11294). The insertion site of AAV into the human genome is called AAVS1. Site-specific integration, as opposed to random integration, will likely result in a predictable long-term expression profile, and may reduce the risk of oncogenic transformation.

So far, eight serotypes of AAV have been identified. The AAV serotypes have different tropisms most likely as a result of their use of different cell-entry receptors. For example, it has been demonstrated that the primary receptor of AAV-2 is HSPG (Heparan Sulfate Proteoglycan) (Summerford, C., and Samulski, R. J., J Virol, 1998; 72:1438-1445) whereas the receptors of AAV-4 and AAV-5 are Sialic Acid based (Kaludov et al., J Virol, 2001; 75:6884-6893). In addition, for AAV-2, both the FGF-receptor (Qing et al., Nat Med., 1999; 5:71-77) and αvβ5-integrins (Summerford et al., Nat Med., 1999; 5:78-82) have been reported to serve the role of co-receptors, although this notion has been challenged (Qiu et al., Nat Med 1999; 5:467-468).

While several AAV serotypes are now under investigation to be used for gene therapeutic applications, AAV-2 is by far the most commonly used. Henceforth, in this disclosure, the term AAV refers to AAV-2 unless stated otherwise. The small (20-25 nm) icosahedral virus capsid of AAV is composed of three proteins (VP1, VP2, and VP3; a total of 60 capsid proteins compose the AAV capsid) with overlapping sequences. The proteins VP1 (735 aa; SEQ ID NO:1; Genbank Accession No. AAC03780), VP2 (598 aa; SEQ ID NO:2; Genbank Accession No. AAC03778) and VP3 (533 aa; SEQ ID NO:3; Genbank Accession No. AAC03779) exist in a 1:1:10 ratio in the capsid. The precise arrangement of VP1, VP2 and VP3 in the capsid is currently unknown because the viral structure showed only VP3 (Xie et al., Proc Natl Acad Sci USA, 2002; 99:10405-10410).

AAV can infect a wide range of different cells due to the rather ubiquitous expression of its receptor HSPG. This property, however, can be either advantageous or detrimental, depending on the specific application. For certain ex vivo applications that use homogenous populations of a specific cell type, the promiscuous nature of AAV is clearly beneficial. By contrast, for most in vivo as well as many ex vivo applications it would be desirable to transfect only specific cells. This would require retargeting AAV by modifying its capsid by adding new targeting information while eliminating its promiscuity, i.e., simultaneously adding a new tropism and eliminating its current one. Whether it is possible to do so efficiently remains, however, to be demonstrated. For example, if AAV indeed requires a co-receptor it is possible that the elimination of HSPG binding would reduce the infectivity of the virus even when a new receptor-binding function is added to the virus capsid. It is therefore more than likely that the effect of eliminating HSPG binding on the viral infectivity will have to be optimized on a case-by-case basis.

Over the past few years several attempts have been made to retarget AAV to specific cell types. Some approaches to retarget AAV have relied on, for example, the binding of covalently coupled antibodies (one of them targeted against the AAV capsid, the other against a surface epitope of a target cell (Bartlett et al., Nat. Biotechnol., 1999; 17:181-186)), binding of ligands to genetically modified AAV capsids (Ried et al., J Virol., 2002; 76:4559-4566) and the binding of avidin-linked ligands to biotinylated AAV (Ponnazhagan et al., J Virol., 2002; 76:12900-12907). The majority of re-targeting experiments, however, have been performed by insertion of a variety of ligands—ranging from peptides to single chain antibodies—into the AAV capsid. In general, these efforts had only modest success (Girod et al., Nat Med., 1999; 5:1052-1056; Grifman et al., Mol Ther., 2001; 3:964-975; Nickel et al., Proc Natl Acad Sci USA, 1999; 96:12571-12576; Rabinowitz and Samulski, Virology, 2000; 278:301-308; Shi et al., Hum Gene Ther., 2001; 12:1697-1711; Wu et al., J Biol Chem., 1994; 269:11542-11546; Wu et al., J Virol., 2000; 74:8635-8647; Zhong et al., J Virol., 2001; 75:10393-10400). For example, Rabinowitz and Samulski (supra and Rabinowitz et al. Virology 1999; 265:274-285), described the production of a relatively small number (43) of AAV mutants containing 12 bp insertions at existing restriction sites in the AAV plasmid. In these studies, peptides were inserted either in all capsid proteins or at least all copies of either VP1, or VP1 and VP2. Shi et al. (Hum Gene Ther 2001; 12:1697-711) constructed a variety of AAV mutants by inserting peptide ligands into the AAV capsid, and found that the residues flanking the actual ligand were important for the viral particle as well as for the transducing titers that could be obtained. Another study tested N-terminal modification of either VP2 or VP3, reporting that viral particles composed of wildtype VP1, VP2, and VP3 proteins as well as VP2 capsid proteins modified with a single chain antibody against CD34 could successfully transduce CD34-positive cells (Yang et al., Hum Gene Ther., 1998; 9:1929-1937). Unfortunately, the titers obtained in the Yang et al. study were extremely low (2×10²/ml). In addition, it cannot be excluded that the low titers reported in this paper were the results of pseudo-transduction (Rabinowitz and Samulski, Virology 2000; 278:301-308; Alexander et al., Hum Gen Ther 1997; 8:1911-20).

Thus, in many cases, modifications such as the insertion of a peptide into the AAV capsid have resulted in reduced transduction efficiency of the mutant AAV, and, while certain peptide insertions into the AAV capsid have had no influence on particle titers, they have completely eliminated virus infectivity (Wu et al., J Virol., 2000; 74:8635-8647).

An interesting approach to retarget AAV has recently been reported independently by Hallek's group (Perabo et al., Molecular Therapy, 2003, 8:151-157) and Kleinschmidt's group (Müller et al., nATURE bIOTECHNOLOGY, 2003, 21: 1041-1046). These authors constructed a library of mutant AAV particles by inserting random peptides into a specific position of the capsid. Selection on target cells then allowed them to identify virus mutants that are able to transduce the target cells While the recently solved crystal structure of AAV (Xie et al., Proc Natl Acad Sci USA, 2002; 99:10405-10) will greatly facilitate the identification of potential positions to insert a ligand without causing detrimental effects, our knowledge of the biology of virus entry and how it is encoded in the AAV capsid is incomplete. Consequently, relying on structural information alone to determine the optimal insertion point for a ligand is most likely insufficient.

Thus, despite significant progress over the last few years, re-targeting of AAV vectors for cell type specific transduction remains an undeniably important but difficult task. Accordingly, there is a need for AAV and other viral vectors having improved targeting abilities for gene therapy and other applications. The invention addresses this and other needs in the art.

### SUMMARY OF THE INVENTION

The present invention is based, in part, on the discovery that certain modifications of the AAV capsid can provide for efficient retargeting of AAV vectors. For example, DNA or plasmid libraries encoding variant AAV capsid proteins with a peptide insert at virtually all possible positions of VP1, VP2, and VP3 can be simply and efficiently prepared as described herein. In addition, functional mosaic AAV vectors comprising both wild-type or variant capsid proteins can be prepared according to the invention.

Accordingly, the present invention provides a DNA library comprising variants of a gene encoding a viral capsid or envelope protein, wherein each variant contains a randomly inserted restriction site. In one embodiment, the library comprises each possible insertion in a variant gene. In another embodiment, the DNA library is a plasmid DNA library. In yet other embodiments, the gene encodes a parvovirus capsid protein or an AAV capsid protein of any AAV serotype. The AAV capsid protein can be, for example, VP1, comprising the amino acid sequence of SEQ ID NO:1; VP2 comprising the amino acid sequence of SEQ ID NO:2; or VP3, having the amino acid sequence of SEQ ID NO:3. The restriction site may be one that results either in blunt ends or overhanging ends upon cutting the library with a restriction enzyme specific for the restriction site. The restriction site may further be flanked with sequences encoding a linker. The linker may, for example, comprise a cysteine residue to promote the formation of a disulfide bond between the flanking linkers.

The invention also provides a library of virus clones, wherein each clone contains a variant of a gene encoding a viral envelope or capsid protein, and wherein each variant contains a randomly inserted restriction site. In one embodiment, the restriction site is flanked by sequences encoding a linker. The linker may, for example, comprise a cysteine residue to promote the formation of a disulfide bond between the flanking linkers. In another embodiment, the library comprises each possible insertion in a variant gene.

The invention also provides for a library of viral clones, wherein each clone contains a variant of a gene encoding a viral envelope or capsid protein, wherein each variant contains a randomly inserted nucleotide sequence encoding a polypeptide sequence. In one embodiment, the library comprises each possible insertion in a variant gene. In one embodiment, the polypeptide is a targeting sequence. In another embodiment, the polypeptide is a peptide that increases the infectivity of viral clones.

The invention also provides for a library of infectious viral particles, wherein each viral particle contains a variant of a gene encoding a viral envelope or capsid protein, wherein each variant contains a randomly inserted targeting polypeptide sequence. In one embodiment, the inserted polypeptide is a targeting sequence. In another embodiment, each viral particle further contains at least one other variant capsid or envelope protein, or at least one other wild-type capsid or envelope protein. The capsid or envelope proteins may be from the same or different viruses. In yet another embodiment, each viral particle contains variant capsid or envelope proteins. The targeting polypeptide may be, for example, a ligand to a receptor expressed by a mammalian cell. In particular embodiments, the targeting polypeptide is a ligand to a receptor expressed by a mammalian cell, the viral particle is a parvovirus, or the viral particle is an AAV of any serotype.

The invention also provides for a method of preparing a plasmid library comprising a viral gene with a randomly inserted restriction site, which method comprises: (a) preparing multiple copies of a first plasmid comprising a first selection marker and a viral gene encoding a viral protein; (b) preparing multiple copies of a second plasmid comprising a second selection marker flanked by transposon sequences, wherein each transposon sequence comprises a restriction site; (c) preparing a first plasmid library by contacting each copy of the first plasmid with a copy of the second plasmid in the presence of a transposase; and (d) selecting a first set of plasmids from the first library that comprise both the first and the second selection markers. In one embodiment, the transposon sequences are Tn7 sequences and the transposase is a Tn7-transposase.

The invention also provides for a method of preparing a library of viral clones, which method comprises transferring each viral gene prepared the method described above into a virus clone, thereby generating a library of viral clones.

The invention also provides for a method of preparing a library of viral clones comprising a heterologous polypeptide sequence randomly inserted in a viral gene, which method comprises treating the plasmid library prepared by the method described above with a restriction endonuclease specific for the restriction site and contacting the treated library with a sequence encoding a targeting polypeptide flanked by the restriction site sequence. In one embodiment, the restriction enzyme generates blunt ends and the oligonucleotide inserted has blunt ends. In another embodiment, the viral gene is a capsid gene or an envelope gene.

The invention also provides for a method of preparing a library of pseudotyped viral particles comprising a variant of a capsid gene or envelope gene, which method comprises expressing the library of infectious clones prepared by the method described above in a host cell transfected with a construct that overexpresses a wildtype capsid protein or envelope protein. In a first embodiment, the virus is a parvovirus. In this embodiment, the virus may be, for example, an AAV of any serotype, and the capsid gene is an AAV capsid gene, and the host cell is infected with a helper virus. In a second embodiment, the host cell is a HEK 293 cell. In particular embodiments, the helper virus is an adenovirus or a herpes virus. In yet another particular embodiment, helper functions are provided by a plasmid. When the virus is an AAV, the AAV capsid gene can encode an AAV VP1 capsid protein comprising the amino acid sequence of SEQ ID NO:1, an AAV VP2 capsid protein comprising the amino acid sequence of SEQ ID NO:2; or an AAV VP3 capsid protein having the amino acid sequence of SEQ ID NO:3, or any combination thereof.

The invention also provides for a method of selecting a virus comprising a variant of a capsid or envelope protein that alters tropism of the virus for a target cell, which method comprises: (a) infecting host cells with a pseudotyped viral particle from the library described above; (b) contacting target cells with viral particles produced from the infected cells of step (a) at a multiplicity of infection of less than 1; and (c) detecting successful infection of the target cells, wherein successful infection indicates that the tropism of the virus is altered such that it infects the target cell. In one embodiment, infection of cells normally infected by the virus is not successful. In another embodiment, the host cells in step (a) express a second capsid protein or envelope protein whereby the viral particles contain a mosaic capsid or envelope. In another embodiment, the peptide can be selected from, e.g., the HA-epitope, the FLAG-epitope, the serpin-ligand, 4C-RGD, L14, LH, LyP-1, Z34C, VEGF, the c-kit ligand, scFv-ACK2, and scFv-ACK4.

The invention also provides for a host cell for expressing a recombinant replication-defective virus, which host cell comprises a first construct encoding a first capsid or envelope protein-encoding gene, a second construct encoding a second capsid or envelope protein-encoding gene which is a variant comprising a targeting polypeptide sequence, and a construct comprising a replication-defective recombinant viral genome comprising packaging sequences and a heterologous gene for a protein of interest. In one embodiment, the first capsid or envelope protein-encoding gene is a wildtype gene. In another embodiment, a ratio of the first construct to the second construct is in proportion to a desired ratio of the proteins in a mosaic viral particle. In another embodiment, more than two capsid-encoding genes are used. In yet other embodiments, the virus is a parvovirus, for example, an AAV, and the helper virus an adenovirus or a herpesvirus. In a particular embodiment, helper functions are provided by a plasmid.

The invention also provides for a replication-defective viral vector comprising a capsid or envelope containing a first capsid or envelope protein, a second capsid or envelope protein that is a variant comprising a targeting polypeptide sequence, and a replication-defective recombinant viral genome comprising packaging sequences and a heterologous gene for a protein of interest. In one embodiment, more than two capsid genes are expressed.

The invention also provides for a method of producing a mosaic replication-defective viral vector, which method comprises: (a) co-transfecting a host cell with a construct comprising a first capsid or envelope protein-encoding gene, a second construct comprising a second capsid or envelope protein-encoding gene which is a variant comprising a targeting polypeptide sequence, and a third construct comprising a replication-defective recombinant viral genome comprising packaging sequences and a heterologous gene encoding a protein of interest; and (b) culturing the host cell under conditions that permit generation of recombinant viral particles comprising a mosaic capsid or envelope. In one embodiment, the first and second constructs are present in a ratio to provide for incorporation of a desired ratio of wild-type to variant capsid or envelope protein in a mosaic replication-defective viral vector produced in the host cell. In another embodiment, more than two capsid genes are expressed. In other embodiments, the virus is a parvovirus, for example, an AAV of any serotype.

The above features and many other attendant advantages of the invention will become better understood by reference to the following detailed description when taken in conjunction with the accompanying drawings.

### BRIEF DESCRIPTION OF THE DRAWINGS

FIG. 1 shows plasmids for linker insertion mutagenesis as described in Example 1. When HEK 293 cells are co-transfected with pDG (a wildtype helper plasmid) and a plasmid that contains a transgene flanked by two AAV-ITRs, it results in the production of recombinant AAV. pDG contains both the AAV Rep and Cap genes as well as the Adenovirus genes required for productive replication in HEK 293 cells. pKS-Cap contains the Cap gene in a derivative of the cloning vector pBluescript that has a SwaI site. pAV2* is an infectious clone of AAV2 that has a ClaI site 3′-of the open reading frame. pGPS4 is the donor plasmid of the linker insertion mutagenesis system from NEB containing the Transprimer with the chloramphenicol resistance.

FIG. 2 shows a transcription map and coding regions of AAV-2. The AAV-2 genome is 4679 nucleotides long. The two open reading frames (Rep and Cap) are indicated by horizontal arrows. The positions of the promoters (map positions p5, p19, p40) are indicated by arrows. Filled boxes indicate coding sequences; nucleotide positions of start and stop codons are shown. V shaped lines indicate introns; nucleotide positions of splice donor and acceptor sites are shown.

FIG. 3 outlines a flow chart of library generation according to the invention.

FIG. 4 shows the construction of the primary library. The donor plasmid (pGPS4, see FIG. 1) is incubated with the acceptor plasmid containing the Cap-open reading frame (pKS-CAP, see FIG. 1) in the presence of TransposaseABC*. This results in the random integration of the Transprimer containing the chloramphenicol resistance gene into the acceptor plasmid. Clones containing a Transprimer insert can then be isolated by selection on ampicillin/chloramphenicol plates.

FIG. 5 depicts one possible clone of the primary library. As indicated, plasmids of the primary plasmid library contain the AAV-Cap ORF and Transprimer insertions. Digestion with PmeI and XmnI results in three fragments. One fragment contains the Transprimer and is of constant length (TP). The length of the other two fragments varies depending on the precise insertion point of the Transprimer within the plasmid.

FIG. 6 depicts one possible clone of the secondary library. As indicated, plasmids of the secondary plasmid library contain the AAV-Cap ORF and Transprimer insertions. Digestion with PmeI and SacI results in three fragments. One fragment contains the Transprimer and is of constant length (TP). The length of the other two fragments (VF) varies depending on the precise insertion point of the Transprimer within the plasmid.

FIG. 7 depicts one possible clone of the tertiary library. As indicated on the left, plasmids of the tertiary plasmid library consist of plasmids with (5aa) peptide insertions (box) into the Cap-region of the infectious clone pAV2*. Digestion with PmeI and XmnI results in two fragment whose length varies depending on the precise insertion point of the peptide within the plasmid.

FIG. 8 shows a selection scheme for viable clones with peptide insertions. In a first step, the tertiary plasmid library is transfected into C12 cells. This cell line stably expresses the nonstructural protein Rep and all capsid proteins. Superinfection with Adenovirus results in the production of the primary AAV library. Because C12 cells express wildtype AAV capsid proteins, this primary library is wildtype-pseudotyped with AAV capsids, and its diversity is limited to the DNA level. This is important because mosaicism on the capsid protein-level of the library would be detrimental for the following steps and might result in a reduction of the complexity of the subsequent libraries. Infection of host cells with the primary library at low MOI and superinfection with Adenovirus or Herpesvirus yields the secondary library that is diverse both on the DNA and the protein level (the capsid proteins containing peptide insertions at different positions). Repetitive selection on target cells, at low MOI and in the presence of Adenovirus, results in a collection of mutant AAV clones that are able to infect the target cells with high efficiency.

FIG. 9A-B show how the capsid-insert could be modified for the AAV-Library. (A) Schematic depiction of pAV2*. The SwaI site is after the stop codon of Rep68/78 and 6 bp before the VP1 start codon. The ClaI site is approximately 90 bp after the VP1 stop codon. Insertions between the stop codon and the ClaI site will result in wildtype capsids. Insertions into the Intron region do not produce viable virus. (B) Schematic depiction of a derivative of pAV2-FseI. The FseI site starts immediately after the VP1 stop codon. The SwaI/FseI fragment of the primary library will be subcloned into this vector, preventing any insertions between the stop codon and the restriction site.

FIG. 10 outlines a detailed analysis of library composition. A plasmid preparation of a library (depicted is a plasmid of a tertiary plasmid library) is first digested with SwaI and end-labeled with phosphokinase and ³²P-γATP. Digestion with BamHI and PmeI will produce two radioactive fragments; a very short BamHI/SwaI fragment and a SwaI/PmeI fragment of variable length depending on the insertion point of the Transprimer. Analysis on a Sequencing gel will then yield a ladder of fragments of variable length. The intensity of each band will represent the relative proportion of the respective insertion mutant in the library. The large labeled plasmid fragment should be well resolved from the fragments of desired length. A similar strategy using ClaI+/−KpnI will yield similar results from the 3′-end.

FIG. 11 shows a two-plasmid system for recombinant AAV (rAAV) production used in Example 6. The rAAV particles are generated by co-transfection of prAAV and pDG. pDG contains both the AAV rep—cap genes as well

FIG. 12 outlines the transfection method to produce the AAV mosaics described in Example 6. The mosaic AAV particles, whose capsid consists of both wildtype and mutant capsid proteins, were produced by co-transfection of a rAAV plasmid together with a wildtype helper plasmid (pDG) and a plasmid carrying capsid proteins with peptide insertions (e.g., pDG-L4 or pDG-L5). pTRFUF11 is a rAAV plasmid that encodes for Green Fluorescent Protein (GPP).

FIG. 13A-C shows viral particle and transducing titers of L4 mosaics. L4-mosaics (encoding for the transgene GFP) were produced by the method outlined in FIG. 12 using the plasmid ratios described in Table 3 and constant amounts of pTRUF11. (A) Viral particle titers (gcp/ml) were determined with Real-Time-PCR (RT-PCR) using a plasmid standard and primers within the GFP open reading frame (ORF). (B) Transducing titers (TU/ml) were obtained by infection of C12 cells with increasing amount of L4-mosaics and co-infection with Adenovirus. The number of transducing units was determined by analyzing the number of GFP-positive cells using FACS analysis. (C) Ratio of gcp/TU, which is a measure for the infectivity of a particular virus preparation.

FIG. 14A-C shows viral particle and transducing titers of L5 mosaics. L5-Mosaics (encoding for the transgene GFP) were produced by the method outlined in FIG. 12 using the plasmid ratios stated in Table 3 and constant amounts of pTRUF11. (A) Viral particle titers (gcp/ml) were then determined with Real-Time-PCR using a plasmid standard and primers within the GFP ORF. (B) Transducing Titers (TU/ml) were obtained by infection of C12 cells with increasing amount of L5-mosaics and co-infection with Adenovirus. The number of transducing units was determined by analyzing the number of GFP-positive cells using FACS analysis. (C) The ratio of gcp/TU, reflecting the infectivity of a particular virus preparation.

FIG. 15 shows the specific transduction of MO7E cells. MO7E cells were incubated for 1 hour on ice with antibody against c-kit. After washing to remove unbound antibody, virus with either wildtype or mosaic capsids encoding for SEAP was added and incubation was continued on ice for an additional hour. After an additional hour at 37° C., the medium was exchanged and the cells were incubated at 37° C. for 48 hours. At that time, the concentration of SEAP in the supernatant was determined using a luminometric assay. The assays were performed in the presence or absence of inhibitors as indicated. The percentages refer to the percent mutant capsid in the viral particles. Hep=Heparin, CD117=antibody against CD117 (c-kit), IgG1 rabbit IgG1, Prot A=soluble Protein A.

FIG. 16 shows the specific transduction of Jurkat cells. Jurkat cells were incubated for 1 hour on ice with antibody against CD29. After washing to remove unbound antibody, virus with either wildtype or mosaic capsids encoding for EGFP was added and incubation was continued on ice for an additional hour. The cells were then incubated at 37° C. for 48 hours. At that time, the percentage of GFP positive cells was determined by FACS analysis. The assays were performed in the presence or absence of Heparin as indicated.

### DETAILED DESCRIPTION OF THE INVENTION

This invention provides DNA libraries, libraries of viral clones and libraries of infectious viral particles and methods of generating these libraries. These libraries are used in the present invention for generating viral particles that have been retargeted to particular cell types by inserting a polypeptide sequence, or retargeting sequence, into the virus's capsid or envelope protein. This retargeting can be done to increase or restrict the range of cells the viral particle infects compared to the tropism of the wildtype virus. In a preferred embodiment, the retargeting allows for a particular cell type to be specifically infected by the viral particle. Such specific targeting will be particularly useful in the advancement of gene therapy because it will allow the gene delivery vehicle (the viral particle) to infect and deliver the therapeutic gene only to those cells intended to be infected, thus decreasing the risk of unwanted side effects from gene therapy and increasing the efficacy of the gene therapy.

In addition, this technique will allow viruses to be generated that can target and infect cell types that were previously resistant to transduction by available vector systems. A recent study has shown that putting the C40 ligand into recombinant AAV viral particles allowed these viruses to infect the previously infection-resistant B cells of chronic lymphocytic leukemia (Wendtner et al. Blood, 2002: 1; 100(5):1655-61). The present invention will allow an infinite number of cell types to be targeted and, if desired, will eliminate native tropism of the viral particle.

An infinite number of cell types can be targeted by the viruses produced in the present invention because the present invention allows the insertion sequence to be inserted into the capsid or envelope protein at every possible site. This random, exhaustive insertion is achieved using linker-insertion mutagenesis, also called linker scanning mutagenesis (Goff, S. P. & Prasad, V. R., Methods Enzymol, 1991; 208:586-603; Barany, F., Proc Natl Acad Sci USA, 1985; 82:4202-6). Linker-insertion mutagenesis also allows the optimal insertion point for specific peptides to be identified. Amplification on target cells and at low MOI will allow the identification of the optimal insertion.

Furthermore, the ideal position can vary from ligand to ligand. As a result, a straightforward method to test all positions to insert a given ligand is highly desirable, and linker insertion is a means by which all positions can be tested.

The linker insertion system used in the present examples is commercially available (New England Biolabs; NEB) and is based on a Tn7 bacterial transposon (Stellwagen, A. E. & Craig, N. L., Embo J, 1997; 16:6823-34; Biery et al., J Mol Biol, 2000; 297:25-37). However, any transposon or linker insertion system can be used to insert retargeting sequences into capsid or envelope proteins.

The NEB system allows the efficient and simple insertion of short linkers (15 bp, i.e. 5 amino acids) into a target sequence. Because the linker sequence is an 8 bp restriction enzyme recognition sequence (PmeI), it also allows the insertion of an additional DNA sequence that can be chosen as desired. This system permits the insertion of the coding sequence for this insertion peptide after every base pair of the sequence encoding for the AAV capsid. Only in four of the six potential reading frames will a particular peptide sequence be inserted into the AAV capsid. This should not be a problem because the subsequent selection procedure will eliminate mutants produced in the two frames that result in the insertion of stop codons.

Not all of the capsid or envelope proteins bearing targeting insertions will be functional. Two possible reasons for non-functionality are 1) defective particle assembly and 2) non-infectivity because the viral particles lack a component necessary for cell entry (for example a ligand for a co-receptor) (Rabinowitz et al. Virology 1999; 265:274-285).

Both of these potential problems are addressed in the present invention by using mosaic viruses: viruses that are composed of a mixture of variants of the same capsid or envelope protein or a mixture of wildtype and variant capsid or envelope proteins. These variants can be retargeted or other mutants of the capsid or envelope protein. For example, a mosaic virus contains wildtype and mutant capsid or envelope protein, or several different mutant capsid or envelope proteins. Non-limiting examples of mosaic viruses are AAV viruses containing L4 capsid protein and either wildtype or retargeted capsid. Another example of a mosaic virus, which contains AAV1 and AAV2 capsids, has recently been reported (Hauck et al. Molecular Therapy, 2003; 7(3):419-425).

As shown in Example 6, the ratio of different capsids in a mosaic virus reflects the ratio of the plasmids encoding these capsids in the viral-producing cell. Thus, the ratio of, for example, wildtype to retargeted capsid, can be altered by changing the number of plasmids encoding wildtype capsid in cell also producing retargeted-capsid. Alternatively, the ratio of different capsid or envelope proteins can be altered by increasing or decreasing expression of the different capsid or envelope proteins by using strong or weak promoters or by controlling protein expression using inducible promoters. A mosaic virus, lacking wildtype capsid or envelope protein, but bearing two or more variants, might lack wildtype tropism, but be able to target the virus to a particular cell type. In the present invention, we demonstrate that mosaics have increased infectious titers when compared to viruses that are made up of mutant capsid proteins alone. Using AAV mosaics, retargeting of AAV by inserting specific peptide-ligands into the capsid will be greatly facilitated and will substantially enhance the utility of AAV as a gene delivery vehicle.

Although the invention is exemplified with AAV2 capsid as a means to retarget the AAV2 virus, envelope proteins of AAV2, capsid or envelope proteins from other serotypes of AAV, or capsid or envelope proteins from other parvoviruses, can be used in the present invention. In addition, the capsid or envelope proteins from viruses from other viral families can be used to retarget their respective viruses, and thus, ultimately, retarget other gene therapy delivery viral vehicles. Other viruses suitable for retargeting and, ultimately as gene therapy delivery vehicles, are well known to those skilled in the art. Such viruses include, but are not limited to, lentiviruses, retroviruses, herpes viruses, adenoviruses, vaccinia virus, baculovirus, and alphaviruses.

For example, a wide variety of alphaviruses may be used as viral vectors, including, for example, Sindbis virus vectors, Semliki forest virus (ATCC VR 67; ATCC VR 1247), Ross River virus (ATCC VR 373; ATCC VR 1246) and Venezuelan equine encephalitis virus (ATCC VR 923; ATCC VR 1250; ATCC VR 1249; ATCC VR 532). Retrovirus include for example HIV, MoMuLV (“murine Moloney leukaemia virus”), MSV (“murine Moloney sarcoma virus”), HaSV (“Harvey sarcoma virus”); SNV (“spleen necrosis virus”); RSV (“Rous sarcoma virus”) and Friend virus.

Various companies produce viral vectors commercially. These viral vectors could ultimately be used, in conjunction with the claimed capsid or envelope protein bearing a retargeting insertion, for gene therapy. These companies include but by no means are limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, and AAV vectors), Norgen (adenoviral vectors), Oxford BioMedica (Oxford, United Kingdom; lentiviral vectors), Transgene (Strasbourg, France; adenoviral, vaccinia, retroviral, and lentiviral vectors), AlphaVax (alphaviral vectors such as VEE vectors) and Invitrogen (Carlbad, Calif.).

Some viruses, such as AAV, will require a helper plasmid in order to be produced because they lack elements essential for viral production. For example, the present invention uses pDG, a wildtype helper plasmid that contains Adenovirus genes (such as E1a, E1b, E2a, and E4), to allow viral particles bearing the retargeted AAV capsid to be produced in HEK 293 cells. Many viruses do not require a helper virus. However, the viruses ultimately used in gene therapy will preferably require a helper plasmid or a producer cell line to be produced because viruses used in gene therapy are preferably replication defective (i.e. are not replication competent), and thus lack all the elements necessary to make viral particles.

### Linker Insertion Mutagenesis

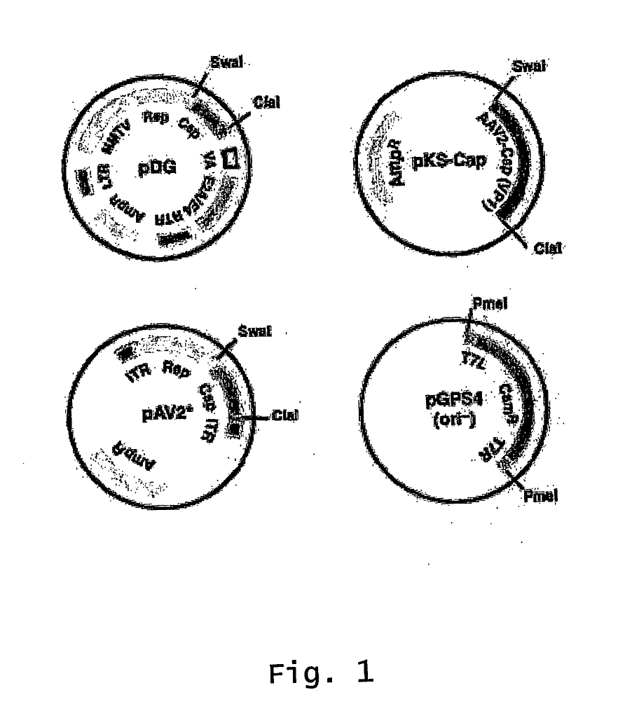

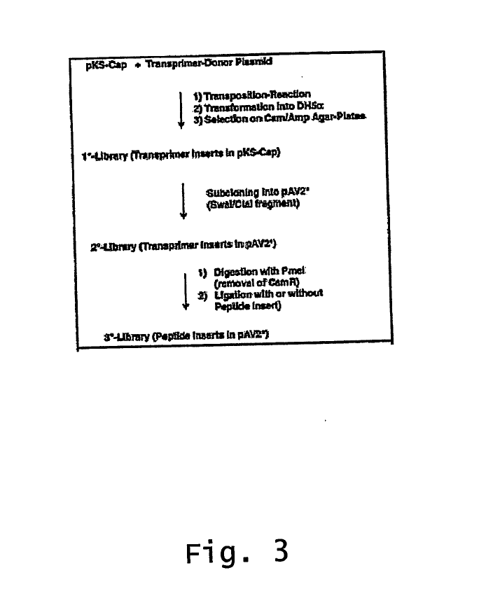

Linker insertion mutagenesis allows the insertion of DNA sequences into target DNA in a random fashion. The system that is commercially available from New England Biolabs is based on the Tn7 bacterial transposon (Stellwagen, A. E. & Craig, N. L., Embo J, 1997; 16:6823-34; Biery et al., J Mol Biol, 2000; 297:25-37). The Tn7-based transposon containing an antibiotic resistance (in our case chloramphenicol resistance) that is flanked by two PmeI restriction sites (8-bp cutter) is encoded by a plasmid that cannot replicate in ordinary laboratory strains of E. coli because it lacks an appropriate origin of replication. Incubation of this Transprimer plasmid (GPS4, FIG. 1) with a target plasmid in the presence of the TnsABC* transposase leads to the insertion of the Transprimer (transposon) into the target sequence (FIG. 3). As a result of target immunity (i.e. the transposase won't transpose into a target sequence already bearing a transposon), in >99% of target sequences only a single Transprimer is inserted into the target sequence. After transformation into a common bacterial strain such as DH5α, target plasmids containing a Transprimer can be selected by growing the bacteria on plates containing both the antibiotics encoded by the Transprimer and the target plasmid respectively. At this stage insertions into important regions of the target plasmid such as the antibiotic resistance gene and its promoter and the origin of replication will be eliminated. Identification of where the transprimer transposed into the target sequence, and thus the location of the inserted site can be determined by sequencing (e.g. using the primers supplied by NEB, PrimerN and PrimerS) or by restriction mapping using the PmeI site.

The present invention is exemplified by the NEB Tn7 transposon system. However, any other suitable transposon or linker insertion mutagenesis system can be used according to the same principles. Furthermore, any restriction enzyme recognition site can be inserted into the viral gene. Selection of the restriction site to be inserted will be dictated by what restriction digest site are present in the viral gene into which transposition will occur and in the plasmid into which the viral gene is cloned.

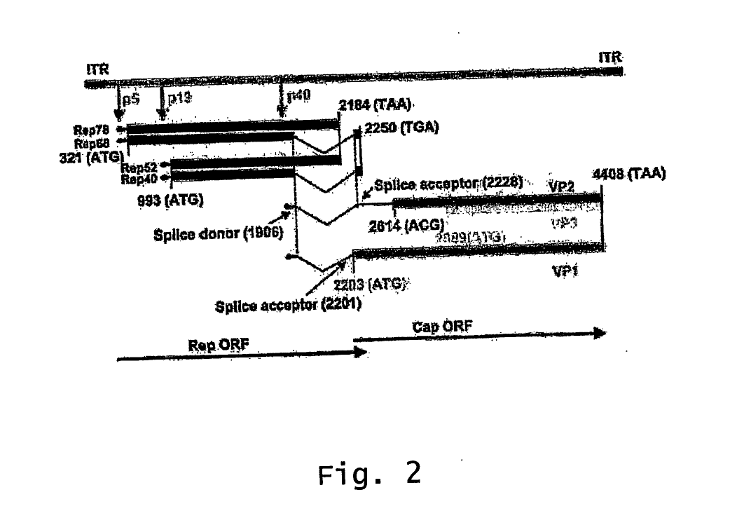

As can be seen from FIG. 2, only two opening reading frames exist in AAV2, one encoding the nonstructural protein Rep that is needed for DNA replication (i.e., terminal resolution), the other encoding the three capsid proteins VP1, VP2, and VP3. The coding sequence of VP3 is common to all these capsid proteins. VP2 and VP3 are encoded by the same transcript but start translation at alternate initiation codons. VP1, on the other hand, is translated from a differently spliced mRNA.

From the genome structure of AAV, it is apparent that any insertion into VP1 will lead to corresponding insertions in VP2 and VP3 if the insertion point is not in the VP1 unique region. If the insertion is in the region common to VP1 and VP2, the ligand will be expressed on both VP1 and VP2. Insertions into VP3, on the other hand, will be displayed on all capsid proteins. Consequently, a library of AAV mutants that carry a ligand insertion at all possible positions within VP1 will cover the entire AAV capsid structure.

Table 1 depicts exemplary amino acid sequences that can be inserted into the viral capsids or envelopes for retargeting purposes. These exemplary insertion sequences are non-limiting examples and include epitope tags and ligands. Any sequence of interest could be inserted into the capsid or envelope protein and be subsequently tested for infectivity of a desired cell type or binding to a desired binding site or receptor. For example, libraries of random peptide sequences could be inserted in order to find optimal receptor-binding sequences.

TABLE 1
Exemplary Insertions for Viral Capids or Envelope Proteins

| | | | | AAV- | |
|---|---|---|---|---|---|
| Sequence or Length | Name | Receptor | Class | Mutant | Cell Line |
| YPVDVPDYA | HA-Epitope | NA | Epitope | Yes³ | 293 |
| (SEQ ID NO: 4) | | | | | |
| DYKDDDKYK | FLAG- | NA | Epitope | Yes³ | 293 |
| (SEQ ID NO: 5) | Epitope | | | | |
| FVLI | Serpin- | Serpin- | Peptide | Yes³ | IB3³ |
| (SEQ ID NO: 6) | Ligand | Receptor | Ligand | | |
| CDCRGDCFC | 4C-RGD | αv-Integrin | Peptide | Yes² | B16F10^{1, 2} |
| (SEQ ID NO: 7) | | | Ligand | | |
| QAGTFALRGDNPQG | L14 | Integrins | Peptide | Yes¹ | B16F10¹ |
| (SEQ ID NO: 8) | | | Ligand | | |
| HCSTCYYHKS | LH | Luteinizing | Peptide | Yes² | OVCAR² |
| SEQ ID NO: 9) | | Hormone | Ligand | | |
| | | Receptor | | | |
| CGNKRTRGC | LyP-1 | Unknown | Peptide | No⁵ | MDA-MB- |
| SEQ ID NO: 10) | | | Ligand | | 435⁵ |
| 34 amino acids | Z34C | IgG* | Peptide | Yes⁴ | 293, HeLa⁴ |
| | | | Ligand | | |
| 148 amino acids | VEGF^{#} | VEGF-Rc | Protein | No⁶ | HUVEC |
| | | (Flk-1) | Ligand | | |
| 273 amino acids | Kit ligand | c-kit* | Protein | No⁷ | 293, HeLa, |
| | | | Ligand | | G1E, |
| | | | | | HCD57, |
| | | | | | MO7e |
| SFv | SC-ACK2 | c-kit* | SFv | No⁸ | 293, HeLa, |
| | | | | | G1E, |
| | | | | | HCD57, |
| | | | | | MO7e |

¹Girod et al., Nat Med, 1999; 5: 1052-6
²Shi et al., Hum Gene Ther, 2001; 12: 1697-711
³Wu et al., J Virol, 2000; 74: 8635-47
⁴Ried et al., J Virol, 2002; 76: 4559-66
⁵Laakkonen et al., Nat Med, 2002; 8: 751-5
⁶Ferrara, N., Nat Rev Cancer, 2002; 2: 795-803
⁷Huang et al., Cell, 1990; 63: 225-33
⁸Ogawa et al., J Exp Med, 1991; 174: 63-71

Table 2 depicts non-limiting examples of flexible linkers for insertion into viral capsids. These flexible linkers can be inserted at the amino and/or carboxy terminus of the inserted sequence and may optimize functionality of the inserted sequence because of the physical space it gives the inserted sequence to achieve its optimal structure.

TABLE 2

Exemplary Flexible Linkers for Insertion Into
Viral Capids

| | |
|---|---|
| Amino-Terminus | C-Terminus |

| | |
|---|---|
| (GlyGlySer)₀₋₃ | (GlyGlySer)₀₋₃ |
| (AlaLeuSer)₀₋₃ | (AlaLeuSer)₀₋₃ |
| (GlyGlySer)₀₋₃Cys | Cys(GlyGlySer)₀₋₃ |
| (SEQ ID NO:11) | (SEQ ID NO:12) |
| (AlaLeuSer)₀₋₃Cys | Cys(AlaLeuSer)₀₋₃ |
| (SEQ ID NO:13) | (SEQ ID NO:14) |

### Mosaic Viral Particles

As described herein, mosaic viruses can be produced that comprise specific proportions of capsid or other viral proteins from at least two different origins or that comprise at least two different variants of the same protein (e.g. two different capsid variants). The different viral proteins can be, e.g., a wild-type and a mutant capsid protein, or two or more different mutant capsid proteins. While the mosaic viruses can be used for any purpose, it has been found that this approach can be applied to fine-tune the targeting properties of a virus.

For example, as described in Example 6, using the AAV L4 mutant, the method of the invention can be used to re-introduce the tropism of a viral particle which capsid proteins have been mutated so that the virus is no longer infectious, by preparing a mosaic virus where a specific proportion of wild-type capsid protein has been introduced. The same principle could also be applied to expand the tropism of a wild-type virus, e.g., by introducing a specific proportion of mutant capsid proteins which have a peptide insert capable of targeting cells which are not part of the wild-type viral tropism. In addition, the method could be used to completely redirect a virus by first deleting the normal tropism of a virus (e.g., such as in the AAV L4 mutant), and then introduce a second mutant capsid protein which have a ligand insert with affinity for a receptor on a different type of cells.

The skilled artisan can easily envision other applications based on the present disclosure, for example, by making a virus that is mosaic in other viral proteins, including envelope proteins. For example, combining viral proteins from different origins can enhance or reduce the infectivity in other aspects than cell targeting or tropism, such as virus stability, nuclear transport of viral nucleic acid, and other features. The method of the present invention also allows for a straight-forward and predictable manner of designing a mosaic virus in that the ratio of the different viral proteins in the final virus can be predetermined by transfecting a host cell with the same ratio of plasmids encoding the different proteins.

### Definitions

The following defined terms are used throughout the present specification, and should be helpful in understanding the scope and practice of the present invention.

Pseudotyping refers to the generation of viral particles bearing a capsid or envelope protein that is from another virus or from a virus bearing a different variant of the capsid or envelope protein. For example, an AAV2 pseudotyped virus is exemplified in the present invention. The exemplified pseudotyped virus of the present invention is a virus with an AAV2 capsid gene, into which a sequence has been inserted, packaged into an AAV2 viral particle bearing wild-type capsid protein. In the present invention, this pseudotyping prevents premature production of mosaic virus.

Replication-defective refers to viral genomes that do not contain a full set of viral genes or that bear mutations that prevent the virus from being able to replicate. Replication-defective viruses are replication incompetent and thus, although capable of infecting cells, cannot replicate once inside the host cell. Replication-defective viruses are the preferred form of viruses to be used for many gene therapy applications.

A packaging sequence is a nucleotide sequence that is recognized by the viral packaging system and thus allows sequences in cis to be packaged into the viral particle. Placement of packaging sequences in cis with heterologous genes of interest allows these heterologous genes to be packaged into infectious, replication-defective viral particles and thus are useful in the production of viral particles for gene therapy.

Mosaic viruses are composed of a mixture of variants or variants and wild-type capsid or envelope proteins. In other words, mosaic viruses contain at least one non-wildtype variant of one or more of their capsid or envelope proteins.

A virus's tropism is defined by the different cell types that it can infect. One preferred embodiment of the present invention is to change the tropism of a virus by retargeting its capsid or envelope protein to a different cell type.

The term overexpression as used herein refers to the expression of a protein at levels much greater than would be expressed under normal, wildtype conditions. Overexpression can be achieved by having many copies of plasmid and/or by using a strong promoter.

The term viral clone refers to a plasmid or construct that contains at least one other viral sequence in addition to the viral gene into which the transposon transposed into. For example, the AAV2 viral clone of the present invention contains the AAV2 Rep sequence and the VP sequences (into which the transposon had transposed) flanked by two ITRs (inverted terminal repetitions). The additional viral sequences present in the viral clone allow the viral gene into which the transposon had transposed into to be expressed and will often provide other components necessary to form an infectious viral particle. Any components necessary for formation of an infectious viral particle not present on the viral clone can be provided on additional helper plasmids.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

### Molecular Biology

In accordance with the present invention there may be employed conventional molecular biology, microbiology, and recombinant DNA techniques within the skill of the art. The general genetic engineering tools and techniques discussed herein, including transformation and expression, the use of host cells, vectors, expression systems, etc., are well known in the art. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

“Amplification” of DNA as used herein denotes the use of polymerase chain reaction (PCR) to increase the concentration of a particular DNA sequence within a mixture of DNA sequences. For a description of PCR see Saiki et al., Science 1988, 239:487.

“Chemical sequencing” of DNA denotes methods such as that of Maxam and Gilbert (Maxam-Gilbert sequencing, Maxam and Gilbert, Proc. Natl. Acad. Sci. USA 1977, 74:560), in which DNA is randomly cleaved using individual base-specific reactions.

“Enzymatic sequencing” of DNA denotes methods such as that of Sanger (Sanger et al., Proc. Natl. Acad. Sci. USA 1977, 74:5463, 1977), in which a single-stranded DNA is copied and randomly terminated using DNA polymerase, including variations thereof well-known in the art.

As used herein, “sequence-specific oligonucleotides” refers to related sets of oligonucleotides that can be used to detect allelic variations or mutations in the gene.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A coding sequence is “under the control of” or “operatively associated with” transcriptional and translational control sequences in a cell when RNA polymerase transcribes the coding sequence into RNA, particularly mRNA, which is then trans-RNA spliced (if it contains introns) and translated into the protein encoded by the coding sequence.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKS plasmids (Clontech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Host cells can further be used for screening or other assays, as described infra.

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. In a specific embodiment, the protein of interest is expressed in COS-1 or C2C12 cells. Other suitable cells include CHO cells, HeLa cells, 293T (human kidney cells), mouse primary myoblasts, and NIH 3T3 cells.

The term “heterologous” refers to a combination of elements not naturally occurring. For example, heterologous DNA refers to DNA not naturally located in the cell, or in a chromosomal site of the cell, or in a virus. Preferably, the heterologous DNA includes a gene foreign to the virus. A heterologous expression regulatory element is such an element operatively associated with a different gene than the one it is operatively associated with in nature.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e., any kind of mutant. For example, in the present invention a variant of a capsid or envelope protein may contain an inserted sequence or may contain nucleotide or amino acid substitutions.

“Sequence-conservative variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 50:667, 1987). Such proteins (and their encoding genes) have sequence homology, as reflected by their sequence similarity, whether in terms of percent similarity or the presence of specific residues or motifs at conserved positions.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 80%, and most preferably at least about 90 or 95%) of the nucleotides match over the defined length of the DNA sequences, as determined by sequence comparison algorithms, such as BLAST, FASTA, DNA Strider, etc. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80% of the amino acids are identical, or greater than about 90% are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc.).

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with 32P-nucleotides or nucleotides to which a label, such as biotin, has been covalently conjugated. Generally, oligonucleotides are prepared synthetically, preferably on a nucleic acid synthesizer. Accordingly, oligonucleotides can be prepared with non-naturally occurring phosphoester analog bonds, such as thioester bonds, etc.

Specific non-limiting examples of synthetic oligonucleotides envisioned for this invention include oligonucleotides that contain phosphorothioates, phosphotriesters, methyl phosphonates, short chain alkyl, or cycloalkl intersugar linkages or short chain heteroatomic or heterocyclic intersugar linkages. Most preferred are those with CH2-NH—O—CH2, CH2-N(CH3)-O—CH2, CH2-O—N(CH3)—CH2, CH2-N(CH3)-N(CH3)-CH2 and O—N(CH3)-CH2-CH2 backbones (where phosphodiester is O—PO2-O—CH2). U.S. Pat. No. 5,677,437 describes heteroaromatic olignucleoside linkages. Nitrogen linkers or groups containing nitrogen can also be used to prepare oligonucleotide mimics (U.S. Pat. No. 5,792,844 and U.S. Pat. No. 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 1991, 254:1497). Other synthetic oligonucleotides may contain substituted sugar moieties comprising one of the following at the 2′ position: OH, SH, SCH3, F, OCN, O(CH2)nNH2 or O(CH2)nCH3 where n is from 1 to about 10; C1 to C10 lower alkyl, substituted lower alkyl, alkaryl or aralkyl; Cl; Br; CN; CF3; OCF3; O—; S—, or N-alkyl; O-, S-, or N-alkenyl; SOCH3; SO2CH3; ONO2; NO2; N3; NH2; heterocycloalkyl; heterocycloalkaryl; aminoalkylamino; polyalkylamino; substitued silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

### EXAMPLES

The following Example(s) are understood to be exemplary only, and do not limit the scope of the invention or the appended claims.

### Example 1

### Linker Insertion Mutagenesis to Modify AAV Capsid

To retarget AAV to specific cell types, it is necessary to modify the AAV capsid. One strategy to do so is to insert ligands that bind to cell-specific surface receptors. This Example shows the production of a plasmid library encoding AAV mutants that carries an insert at every possible position of the AAV capsid (at least on the DNA level), theoretically allowing the assembly of all possible peptide insertion mutants (2205) of AAV for this specific peptide. Here, the insert was a 5-residue peptide having one of the following sequences:

| | |
|---|---|
| PCLNS | (SEQ ID NO:15) |
| GCLNT | (SEQ ID NO:16) |
| LFKHN | (SEQ ID NO:17) |

The same methodology used in the instant experiment can be repeated with a peptide insert which is a receptor ligand. A selection procedure on a cell type expressing the receptor would then allow for the isolation of mutants with particularly high infectivity. For example, multiple rounds of selection at low multiplicities of infection of 0.01 to 0.1 on the desired cell line would identify AAV mutants with comparatively high infectivity for the specific cell type.

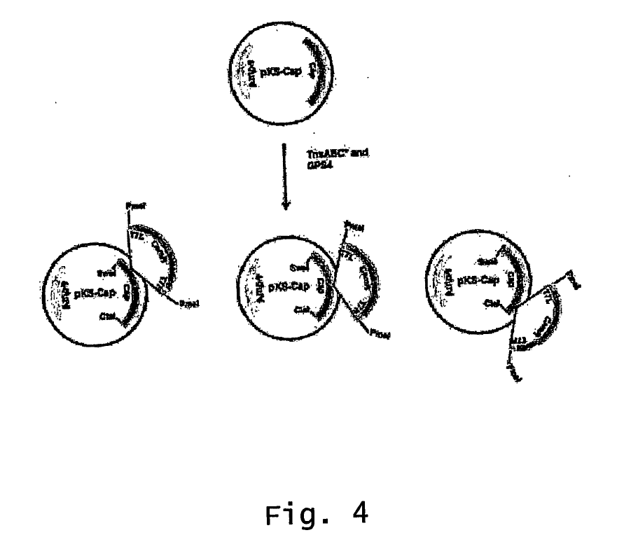

First Plasmid Library. First, the coding sequence of VP1 into a general cloning vector was cloned by excising the Cap region from the plasmid pDG (FIG. 1) (Grimm et al., Hum Gene Ther, 1998; 9:2745-60) with SwaI and ClaI and ligating it into a modified version of pKS (Stratagene) cut with the same enzymes to produce pKS-Cap (FIG. 1). Next, a primary plasmid library was constructed by incubating the donor plasmid GPS4 (FIG. 1) with the target DNA pKS-Cap (FIG. 1) in the presence of TransposaseABC* (FIG. 4). Transformation of the plasmid mixture resulting from this transposition reaction into a bacterial strain such as DH5α and subsequent selection on Chloramphenicol and Ampicillin containing plates allowed the isolation of a library of plasmids. These plasmids contained the entire sequence of pKS as well as a Transprimer region from the donor plasmid. A substantial fraction of this plasmid library will be composed of plasmids with an insertion within the Cap region; a minority, however, will contain insertions into the nonessential regions of the vector backbone.

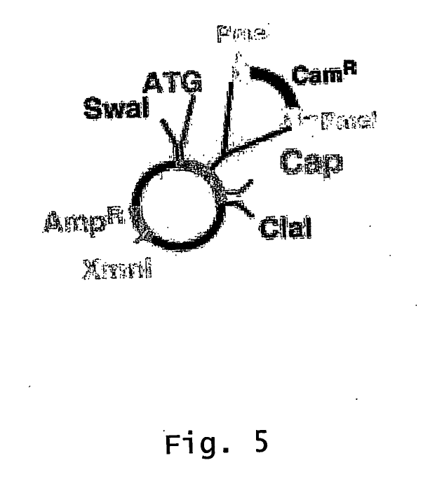

Because the Cap coding sequence is later subcloned into a different vector, Transprimer insertion into the vector backbone will be eliminated at that step. The plasmid library that was obtained in this manner had 22,000 clones. From the size of the plasmid (5.3 kb), it was apparent that each possible insertion point was represented at least 4 times. That the plasmid library contained plasmids with insertions of the Transprimer at different positions of the target sequence was demonstrated by a restriction analysis with enzymes XmnI and PmeI. Briefly, plasmid DNA of 9 individual clones was digested with PmeI and XmnI and analyzed by agarose gel-electrophoresis and ethidium-bromide staining. This digestion was expected to result in three fragments. One of those fragments should be of constant size because it encompasses the Transprimer region; the other two fragments should be variable in size, depending on the insertion of the Transprimer in the plasmid. The stained gels resulting from the restriction analysis clearly showed that the plasmid library contained individual clones. One possible clone is depicted in FIG. 5.

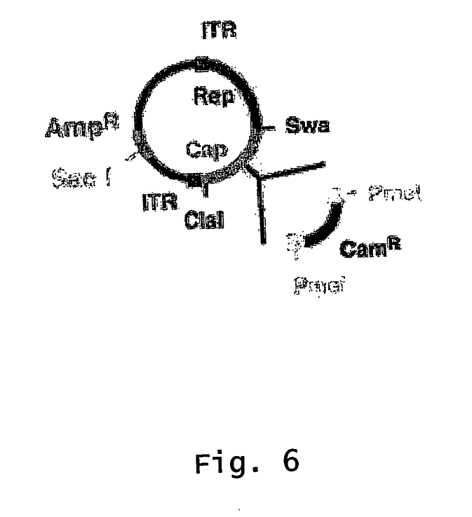

Second Plasmid Library. Next, the Cap coding region of the primary plasmid library was excised and subcloned into an infectious clone plasmid pAV2*. This produced a secondary plasmid library that still contained the Transprimer, including the chloramphenicol resistance gene. This plasmid library consisted of 14,000 clones. The smaller number of complexity at this step was expected because part of the primary plasmid library consists of insertions into the plasmid backbone. Again, restriction analysis (SacI/PmeI) was used to analyze if the plasmid library contained individual clones. Briefly, plasmid DNA of individual clones was digested with SacI and XmnI and analyzed by agarose gel-electrophoresis and EtBr-staining. The stained gels clearly demonstrated that individual clones were present in this plasmid library. One possible clone is depicted in FIG. 6.

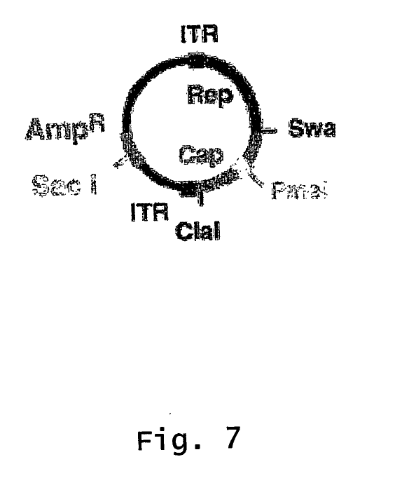

Tertiary Plasmid Library. Finally, the Transprimer was removed from the secondary plasmid library to produce the third and final plasmid library (see FIG. 3). In this last step the number of clones remained more or less constant at 15,000. Individual clones were analyzed by restriction analysis with SacI/PmeI. Briefly, plasmid DNA of individual clones was digested with PmeI and SacI and analyzed by agarose gel-electrophoresis and ethidium bromide staining. The results demonstrated individual clones. To analyze the plasmid library in more detail, methods with higher resolution (e.g., sequencing) can be used. One possible clone is depicted in FIG. 7.

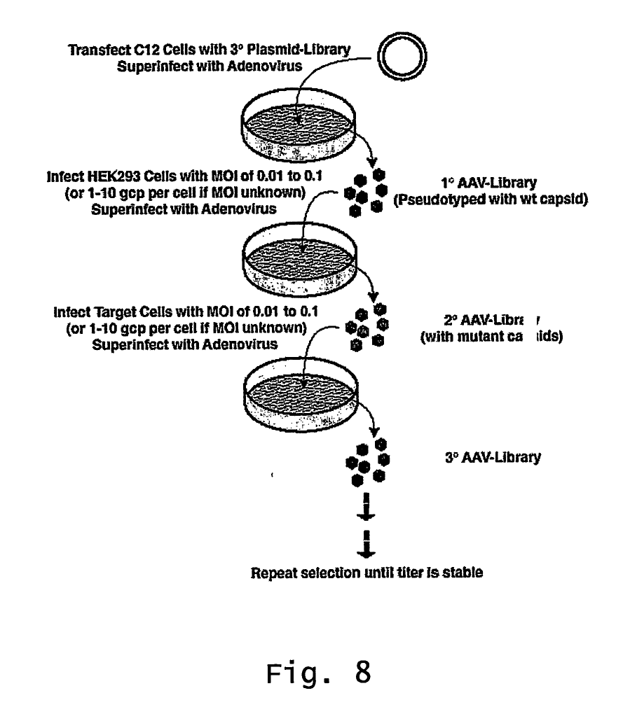

Prevention of Mosaic Viruses. This third plasmid library, which contains 15 bp (i.e. 5 aa) insertions in the capsid, was then used to transfect C12 cells (followed by Adenovirus superinfection; step one in FIG. 8). Transfection of cells, especially by calcium phosphate precipitation, will result in the uptake of more than one plasmid into the same cell. In HEK 293 cells it is expected that this would cause the production of mosaic AAV particles that are composed of capsid proteins containing various insertions. In addition, the viral DNA encapsulated does not necessarily correspond to any of the capsid proteins. While controlled production of specific mosaic viruses may be applied to further increase targeting efficiency (see Example 2), at this step, it should be avoided.

Therefore, to prevent uncontrolled, premature formation of mosaic capsids, we pseudotyped the primary AAV library with wildtype-AAV capsids by transfection into C12 cells (FIG. 8), a cell line that overexpresses AAV-Cap (and Rep). Next, HEK 293 cells were infected with this library at an MOI of 0.1. After superinfection with Adenovirus, this yielded a secondary AAV library. This library contains AAV virions that have mutant capsids encoded by their encapsidated genomes. This selection step was repeated once to produce a tertiary AAV library with a titer of (1.5×10¹⁰ gcp/ml). An initial analysis of this library indicated that it contained substantial amounts of wildtype AAV. The reason for the presence of wildtype AAV in this library can be largely attributed to an AAV-contamination of our Adenovirus stock. In addition, it is possible that insertion of the Transprimer into a short region of DNA located between the stop codon of VP3 and the ClaI site resulted in plasmids that upon transfection into C12-cells yield wildtype AAV. These undesired features can easily be avoided by repeating these experiments with AAV-free Adenovirus and a modified version of the plasmid pKS-Cap.

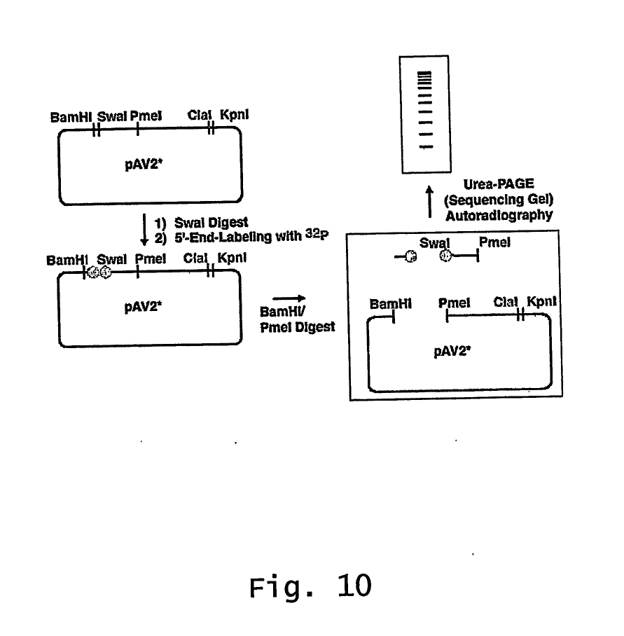

In summary, this Example demonstrates the feasibility of producing a plasmid library of infectious AAV-clones with 15 bp insertions in the capsid coding region; the library is of sufficient complexity to assure that insertions after each possible base pair are represented (FIG. 7). In a two-step procedure (step 1 and 2 of FIG. 8), this yielded a library of AAV particles that contained 5 amino acid insertions at all the possible positions of the capsid that are able to produce intact viral particles. If desired, an additional peptide sequence may be inserted in the final step of the plasmid library production (FIG. 3). From the final plasmid library, AAV libraries containing the 5 aa insertions from the Transprimer insertion or libraries containing the desired ligand are generated. Viral clones with high infectivity are then identified by selection on the chosen target cells (FIG. 8). Individual clones of the plasmid libraries are analyzed by restriction analysis as described above. In addition, individual clones are sequenced and an detailed analysis of the library performed as outlined in FIG. 10.

### Example 2

### Restriction to Capsid Insertions

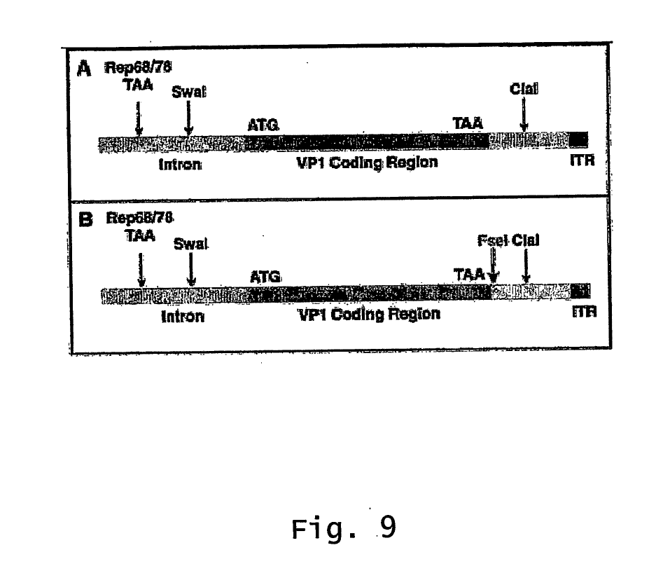

As described in Example 1, the Cap region of AAV was subcloned into pKS to produce the initial plasmid library by excising VP1-coding region from pDG with SwaI/ClaI (pKS-Cap, FIG. 1). Similarly, an infectious clone with these restriction sites was generated by inserting a ClaI site into pAV2 (pAV2*, FIG. 9). To produce the secondary plasmid library containing the Transprimer insertions in pAV2*, this SwaI/ClaI fragment was excised from the plasmids of the primary library (FIG. 4) and inserted into pAV2* digested with the same enzymes. With this procedure, this secondary plasmid library will contain insertions not only in the VP1 coding region but also between the SwaI site and the ATG start codon of VP1 as well as between the TAA stop codon of VP1 and the ClaI site. Previous experiments have shown that even small changes, such as single point mutations, in the region between the SwaI site and the VP1 start codon, will result in non-infectious clones. In addition, the ATG is only 6 bp after the end of the SwaI site. In addition, insertions into the region between the VP1 stop codon and the ClaI site, might produce infectious clones. These clones would produce AAV particles that have mutant genomes but a wildtype capsid. It is possible that such viruses will have a growth advantage over viruses that contain peptide insertions in their capsid.

To eliminate this potential complication, modified versions of pKS-CAP, pAV2*, and pDG have been produced that contain a FseI restriction site immediately after the VP1 stop codon (FIG. 9). This enables subcloning of a SwaI/FseI fragment from the primary plasmid library into pAV2-FseI. After step 3 of the procedure outlined in FIG. 3, the tertiary library now derived can contain only infectious clones with insertions in the capsid.

### Example 3

### Linker Insertion Mutagenesis with Peptide Ligands

This Example outlines the preparation and screening of AAV plasmid libraries with ligand inserts at all possible sites. Primarily, HA-epitopes as well as an Integrin-binding ligand called L14 (Girod et al., Nat Med, 1999; 5:1052-6) are prepared. Mutant AAV that present this ligand have been generated previously and have been demonstrated to be able to transduce the Integrin expressing mouse melanoma cell line B16F10. The transducing titers reported, however, are comparatively low (Girod et al., Nat Med, 1999; 5:1052-6). The instant experiments will determine whether better insertion sites are available.

Next, optimal insertions into the AAV capsid are determined for a variety of peptides and protein ligands of various sizes. Table 1 (above), lists peptide and protein ligands of interest. The ligands and epitopes listed in Table 1 include both sequences for which AAV mutants have been reported in the literature as well as sequences for which no mutants have been published. The instant experiments will determine whether better insertion sites are available. In addition, because of the diverse nature of the insertions (especially as it refers to short peptides), it will be determined whether there is one specific insertion point or if the optimal insertions point depends on the peptide sequence inserted into the capsid. Obviously, a general insertion point would be of particular interest. Most AAV mutants with the ligands listed above will be of considerable biological interest. In particular, the determination of an ideal insertion point for a single-chain Fv-fragment (scFv) against the c-kit antigen is of significant value because the overall folding of all scFvs is very similar. It is therefore likely that the optimal insertion point for many, if not all, scFvs will be the same.

Briefly, plasmid libraries with insertions of ligands at all possible positions in the AAV are produced starting from a secondary plasmid library (FIG. 6 and FIG. 7), which can be considered a master restriction-site library. This library is digested with PmeI and a double stranded oligonucleotide or blunted DNA fragment coding for the ligand inserted. Individual clones of this tertiary plasmid library are then analyzed by restriction digest as described above. A detailed analysis of the library is performed as described in FIG. 10.

### Example 4

### Linker Insertion Mutagenesis with Flanking Sequences

This Example outlines the analysis of the effect of linker sequences on viral titers. Specifically, the influence of variable numbers of short flexible linkers such as Gly-Gly-Ser on either side of the peptide ligand on viral titer and infectivity are investigated. In addition, the effect of the introduction of twin cysteine residues adjacent to the ligand are examined. These cysteine residues allow for the formation of a disulfide bridge and, as a result, limit the available conformations of the inserted peptide. Such conformational restriction often leads to higher affinities of a specific peptide sequence for its receptor and has been exploited previously in the identification of peptide ligands via phage display (Hoess et al., J Immunol 1994; 153:724-9). The combinations of linkers that we will test initially are listed in Table 2 (above).

A library composed of all possible combinations of peptide and linker is prepared to select for the best possible permutation. To produce such libraries, a mixture of the double stranded oligonucleotides encoding for the desired peptide and the possible linkers are inserted. Pairing each of the amino-terminal linkers with each of the carboxy-terminal linkers on the same row of Table 2 results in 16 possible combinations. A library is then prepared to select the most infectious AAV mutants using the procedure outlined in FIG. 8. Alternatively, the 4 possible plasmid libraries of rows 1 through 4 are produced and then pooled before selection on target cells. This method has the advantage that it reduces substantially the work needed to screen for all possible linkers. It is also possible to produce a library that contains all possible amino-terminal linkers (of all rows) paired with all possible carboxyl-terminal linkers (of all rows). Such libraries may, however, become quite large (with the possibilities listed above it would be ˜65,000-times the complexity of the starting library (15,000 clones), i.e. ˜1×10⁹).

### Example 5

### Linker Insertion Mutagenesis with Alternative Restriction Sites

This Example describes the use of other restriction sites than PmeI. The restriction site introduced with the method described in Example 1 is a blunt end cutter (PmeI). Ligations with vectors and inserts that are blunt ends can be more challenging than the ligation of DNA fragments that have overhangs, especially for the production of high complexity libraries with ligand insertions. Since it is not possible simply to exchange the PmeI sites in the Transprimer with suitable 8 bp restrictions sites that produce overhangs because of sequence restrictions imposed by the transposon system, the following methodology is employed.

A tertiary plasmid library (i.e., a secondary restriction-site master library) is produced by inserting a 9 bp linker that includes a NotI site (an 8 bp cutter that produces a 5′ CCGG overhang) that is neither present in pAV2* (or pAV-FseI) nor in pKS-Cap. A tertiary library with a NotI site after each nucleotide of the VP1 ORF can serve as a master library for the generation of ligand-containing tertiary libraries. In addition, modified master libraries with a NotI site as well as (a library of) linker amino acids (Table 2) are ideal starting libraries to insert the coding region of choice.

### Example 6

### Retargeting AAV-2 Using Mosaic Vectors

This Example demonstrates that mosaic viruses composed of both wild-type and mutant viral capsid proteins can have increased infectivity when compared with viruses that are made up entirely by mutant capsid proteins. This method can be applied to re-target AAV to specific cell types or to increase its infectivity.

In a first step, it was determined if it was possible to render an otherwise non-infectious virus that forms intact capsids infectious by replacing defined amounts of mutant capsid proteins with wild-type proteins. This was tested in AAV mutants developed by Muzyczka and co-workers (Wu et al., J Virol., 2000; 74:8635-8647), which mutants have HA-epitopes inserted in the capsid and produce full capsids but are non-infectious. The HA-epitopes are expressed on the surface of the capsid, allowing purification, detailed characterization, and confirmation that the infectious viral particles are composed of both mutant and wildtype proteins. The capsids of these AAV mutants, called IA and L5 (Wu et al., supra), carry HA-epitope (YPVDVPDYA; SEQ ID NO: 5) insertions at amino acids 522 and 553 respectively (Wu et al., supra). The reason for the lack of infectivity of the mutant L5 is unknown, whereas the lack of infectivity of L4 is a result of its impaired ability to bind to HSPG (Wu et al., supra).

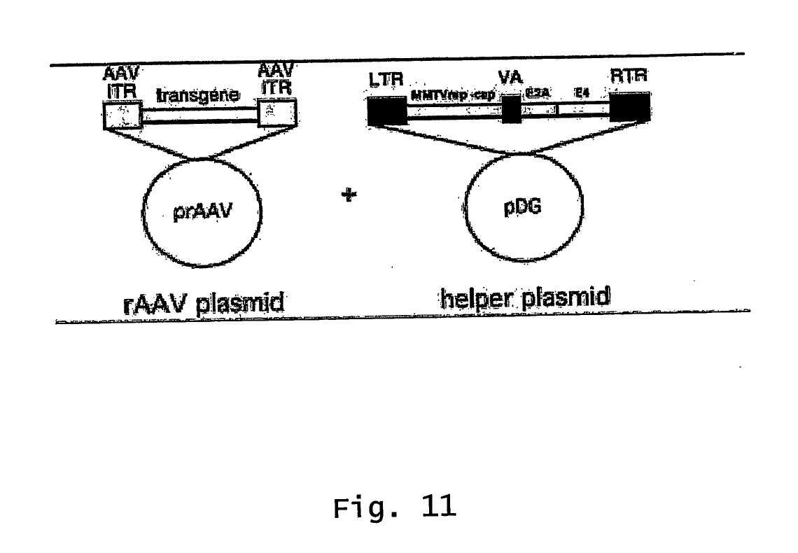

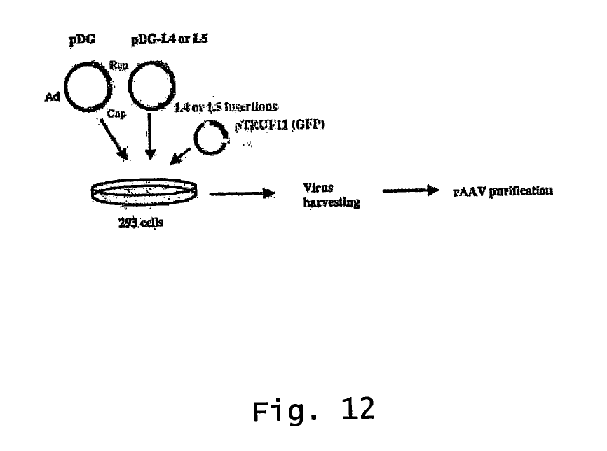

FIG. 11 shows the two-plasmid system for recombinant AAV (rAAV) production utilized. Briefly, rAAV particles are generated by co-transfection of prAAV and pDG. pDG contains both the AAV rep—cap genes as well as the Ad genes required for productive replication in 293 cells. To test the principle of AAV mosaicism, the experiment outlined in FIG. 12 was performed. For this purpose, the mutated cap genes were subcloned into pDG generating pDG-L4 and pDG-L5.

HEK 293 cells were co-transfected with a constant amount of total helper plasmid (i.e., the sum of pDG and pDG-L4 or pDG-L5) but varying percentages of pDG-L4 and pDG-L5 DNA, starting from 0% up to 100% (see Table 3). To keep the amount of helper plasmid constant, the transfections were supplemented with an appropriate amount of pDG DNA (Table 3). The amount of pTRUF11 DNA used for transfection was maintained constant. After harvesting the viruses, they were purified by Iodixanol gradient and heparin affinity purification (where applicable). The number of genome containing particles was determined by Real-Time PCR, and the transducing units by FACS (GFP expressing cells).

TABLE 3
Transfection Conditions

Percentage of DNA
used transfection

| | | | | | | |
|---|---|---|---|---|---|---|
| pDG-L4 or pDG-L5 | 0 | 10 | 25 | 50 | 75 | 100 |
| Pdg | 100 | 90 | 75 | 50 | 25 | 0 |

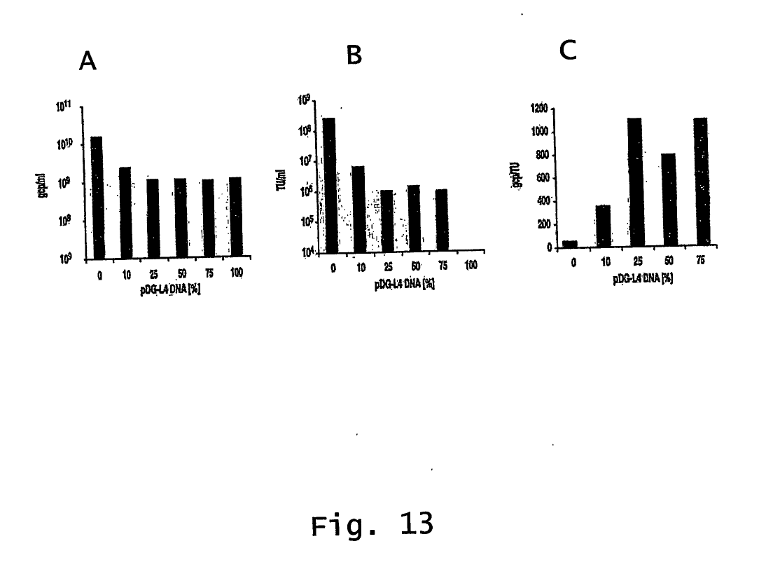

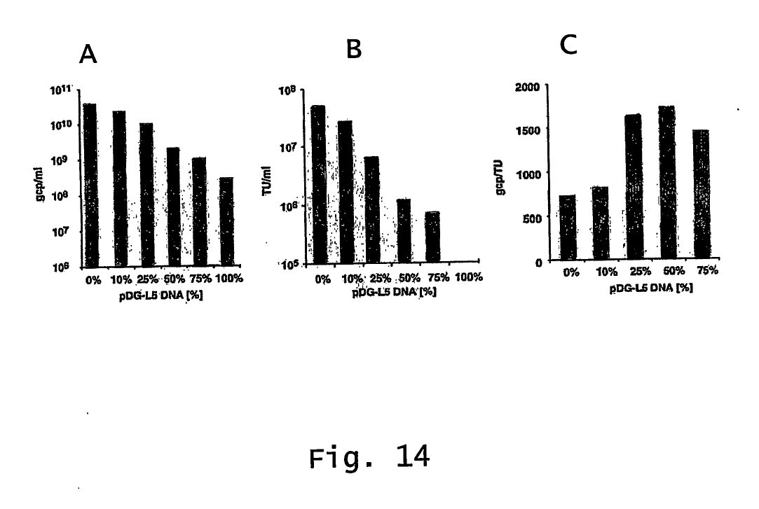

As described by Wu et al., supra, 100% mutant rAAV (L4 and L5 insertion mutants) were able to efficiently form virus particles at about one to two logs lower than the wild-type, but these viruses were not infectious. Interestingly, even when only small amounts of wild-type plasmid were added to the transfections, likely resulting in very limited amounts of wild-type protein within the capsid, the mosaic rAAV regain infectivity. For L4 mosaic rAAV, the GCP/TU ratio was 6 to 20 times higher than the wild-type rAAV (FIG. 13), whereas for L5 the wild-type virus and the 10% L5 virus exhibited similar infectivity (FIG. 14). One possible explanation for the overall lower infectivity of L4-rAAV when compared with L5-rAAV is the fact that the site of insertion of the HA tag in L4 disrupts the heparin binding domain whereas L5 does not. Very similar results were obtained when the virus was not purified but instead cell lysates were used for the experiment.

To characterize the viruses, the purity of the purified viruses was assessed by SDS page and silver staining. Preparations of greater than 90% purity were routinely obtained. To demonstrate the presence of the HA-peptide in the viral capsids, the viral preparation was analyzed by Western blot using monoclonal antibodies against the AAV capsid (B1; Wobus et al., J Virol., 2000; 74:9281-9293) and against the HA epitope (16B12; Covance Research Products, Denver, Pa.). Capsid proteins with HA epitope insertions could be seen for all capsid proteins. Western blot with B1-antibody of a virus preparation of 25%-L4 virus revealed the presence of VP3 of wildtype size as well as a band that migrated at a slightly higher molecular weight. On longer exposures, similar bands could be observed for VP1 and VP2. That this larger band represented VP3 capsid proteins with an HA insertion was demonstrated by the fact that the same band was recognized by the anti-HA monoclonal antibody 16B12. Furthermore, the intensity of the two bands was consistent with a ratio of 3 to 1 of VP3 vs L4-VP3. Thus, the ratio of wildtype to mutant VP3 detected by B1 was consistent with a viral particle composition of 75% and 25% VP3 vs L4-VP3A. This indicated that the ratio of plasmids during the transfection resulted in similar proportions of the resulting proteins in the viral particles, at least for L4-rAAV.

To analyze the integrity of the viral particles, negative staining electromicroscopy (EM) was conducted. Briefly, wildtype and 25%-L4-rAAV virions were adsorbed to parlodion-coated grids and negatively stained with 1% Uranyl acetate. Both wildtype-rAAV and 75% L4-rAAV preparations contained both empty and full intact viral particles. Together, these results showed that the procedure outlined in FIG. 12 generated mosaic rAAVs that were infectious, in contrast to the non-infectious homogeneous rAAVs that carried the same peptide insertion in all capsids.

To rigorously exclude the possibility that the infectious particles observed in experiments with bona fide mosaic viruses consisted of virions with a wild-type-capsids, HA-modified mosaic viral particles were immunoprecipitated with anti-HA antibodies coupled to beads. Briefly, either “wildtype” or 75%-L4-rAAV mosaic virus particles were incubated overnight with anti-HA (16B12) or anti-AU1 beads (negative control beads; Research Genetics). After extensive washing equal amounts of the pellet and supernatant were analyzed by SDS-PAGE and Western-Blot (anti-VP3, B1). Analysis by Western-Blot using an antibody against the capsid-protein VP3 revealed that all the viral particles in the preparation could be precipitated with this procedure. On longer exposures VP1 and VP2 were visible in all positive samples. These data supported that the infectious viral particles are indeed mosaic virions. It does, however, not rigorously prove it. To exclude the possibility that virions with wild-type capsids remained in the supernatant but that the amount was below the detection limit of the Western Blot, we eluted the virions bound to the beads with HA-peptide. The eluted particles had to be mosaic as they could be precipitated with anti-HA antibodies. Then, these virions were tested for infectivity by infecting C12-cells in the presence of Adenovirus. C12 cells were infected with either non-purified 25%-L4-rAAV Mosaics (expressing GFP) or with virus eluted from beads after Immuno-Precipitation with anti-HA antibody beads (16B12). The fluorescence micrographs obtained demonstrated the infectivity of both viral preparations. Thus, the presence of GFP-expressing cells clearly demonstrated that these particles were infectious.

These results demonstrate that mosaic viruses can increase the infectivity of virions with peptide insertions in their capsid, and that noninfectious viral mutants can be rendered infectious if mosaic viruses are produced that also contain wild-type capsid proteins.

### Example 7

To demonstrate that AAV Mosaics are useful tools to alter AAV tropism an AAV mutant originally developed by Hallek and co-wokers (Ried, M. U., J. Virol. 2002 76:4559-4566) was employed. The mutant developed by Ried et al. contains an insertion of a short fragment of Protein A into the AAV capsid at position 587. As a result, this AAV mutant can bind antibodies to potential receptors for viral entry. Indeed, this group demonstrated that it is possible to transduce specifically certain cell types with this mutuan AAV and appropriate antibodies. They showed, for instance, that they can selectively transduce the c-kit positive human erythroleukemia cell line MO7E. Unfortunately, however, the viral particle titers that could be produced with this mutant capsid were more than an order of magnitude lower than virus generated with wildtype capsid. Maybe even more important, the infectious titers achievable with the AAV mutant were not higher than the titers achievable with recombinant AAV that has a wildtype capsid. These results show that the insertion of the Protein A fragment into the viral capsid results in significant deleterious effects on particle as well as infectious titers.

In light of these results, AAV mosaics were generated whose capsids are composed of both wildtype capsid proteins and capsid proteins with a Protein A fragment insertion at position 587 were generated. The binding domain of Protein A that was used is called Z34C and was first described by Starovasnik et al. in Proc. Natl. Acad. Sci., 1997, 94: pp 10080-10085. Its amino acid sequence is:

FNMQCQRRFYEALHDPNLNEEQRNAKKSIRDDC (SEQ ID NO 18). AAV mosaics that were composed of 25% and 50% mutant capsid (as determined by Western Blot) at near wildtype titers were produced. As expected, it was not possible to generate virus that is composed entirely of mutant capsid proteins at satisfactory levels. The particle titers obtainable were at least 1000-fold lower than virus particle titers of recombinant AAV with wildtype capsid.

The percentage of mutant capsid protein was determined as follows: Samples of purified mosaic AAV were loaded on a 7.5% SDS-polyacrylamide gel, and transferred onto a Hybond-P membrane (Amersham). Then, the proteins were probed with the above-described monoclonal antibody B1 (Research Diagnostics). The bands were visualized by a Horseradish-Peroxidase-coupled goat anti-mouse secondary antibody and the ECL Plus Western Blotting Detection System (Amersham). The percentage of mutant capsid proteins vs wild type proteins was estimated based on the relative intensity of the bands on the film.

First, these mosaic AAV preparations—whose genome encodes secreted alkaline phosphatase (SEAP)— were tested on the human megakaryocytic erythroleukemia cell line MO7E both in the presence and absence of antibodies against c-kit. As additional controls was measured the transduction in the absence or presence of heparin, rabbit IgG or Protein A.

The percentages of mutant capsid protein tested were: 10%, 25%, 50%, 75% and 100%. It was not possible to produce significant high enough titers of 100% mutant virus to test transduction efficiencies. Although 50% mutant capsid was also effective for retargeting, higher amounts of viral particles must be used to get the same transduction efficiency. In other words, the ratio of genome containing particles to transducing units is higher. The 75% mutant capsid can be considered as inefficient as an even larger amount of viral particles has to be used.

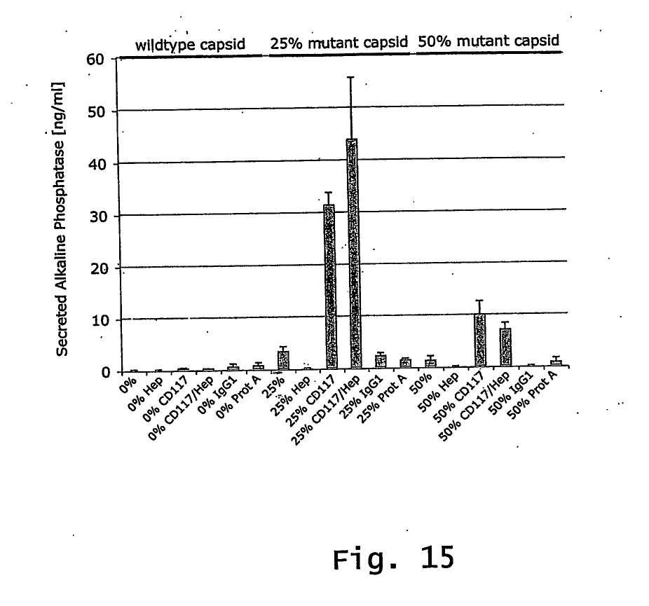

In line with the results reported by Ried et al., recombinant AAV with a wildtype capsid was unable to transduce significantly MO7E cells in either the presence or absence of antibody against c-kit and/or inhibitors (FIG. 15). Similarly, AAV mosaics composed of either 25% or 50% mutant capsid proteins were unable to transduce MO7E cells in the absence of antibody against c-kit both in the presence and absence of Heparin. If, however, antibody against c-kit was present, substantially higher transduction than transduction with virus with a wildtype capsid could be observed both in the presence and absence of Heparin. This transduction could be inhibited by adding either rabbit IgG or Protein A, both competitive inhibitors (FIG. 15.) These results demonstrate that—using AAV mosaics—it was possible to transduce specifically and efficiently MO7E cells.

The transduction efficiency of the 25% mosaic virus was about 10-fold higher in the presence than in the absence of antibody and more than two orders of magnitude higher than wildtype. Hallek and co-workers, using virus entirely composed of mutant capsid, were only able to achieve transduction levels equivalent to wildtype capsid virus.

The fact that the 50% virus is less infectious (3-fold lower) than the 25% virus—despite having presumably higher levels of antibody bound—supports the notion that the mutant capsid is deleterious to the infectivity of virus containing a Protein A fragment insertion at position 587.

The results just described do not, however, rigorously prove that higher infectious titers can be achieved with mosaic viruses—although they demonstrate higher transgene expression. To demonstrate this and to extend these results to additional cell lines, in a second set of experiments, Jurkat cells and GFP as a transgene were used.

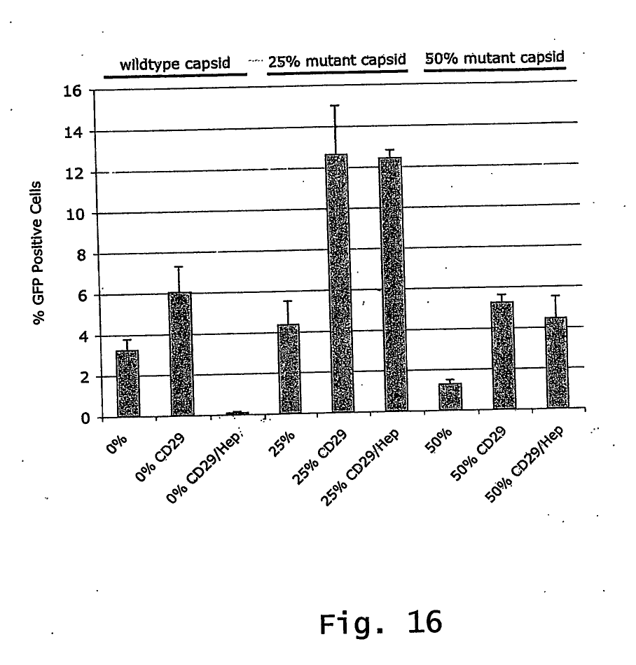

As can be seen from FIG. 16, using 25% mosaics were obtained infectious titers that were about 2-3 times higher than virus with a wildtype capsid. Importantly, in the presence of Heparin the infectivity of the virus with wildtype was completely eliminated whereas the infectivity of the 25% mosaic in the presence of antibody against CD29 was almost identical indicating the specificity of transduction. The introduction of a point mutation into the virus capsid that eliminates HSPG binding should, therefore, allow us to specifically transduce Jurkat cells.

Again, the fact that the 50% virus was less infections (2-fold lower) than the 25% virus—despite having presumably higher levels of antibody bound—supports the notion that the mutant capsid is deleterious to the infectivity of virus containing a Protein A fragment insertion at position 587. Furthermore, the infectious titers that were obtained with 25% mosaic was about 100,000 times higher than the titers reported by Hallek and colleagues using the all mutant virus, again arguing for the advantage of using mosaic viruses.

In light of the above results, the effective amounts of mutant capsid protein for use in the present invention is broadly up to about 50% mutant capsid protein and ranges between about 10% and about 50%, and preferably between about 10% and about 25%.

In summary, it was not possible to achieve significant particle titers of virus with an all-mutant capsid. The particle titers of the mosaic viruses, on the other hand, were similar to those obtained with virus with a wildtype capsid. Furthermore, the transgene expression with 25% mosaic virus was about 100-fold higher than virus with wildtype capsid in the absence of Heparin and >100,000-times higher in the presence of Heparin. In addition, the titers are at least five orders of magnitude higher than the infectious titers reported by Hallek and colleagues using all mutant virus.

These results demonstrate that AAV mosaics are useful tools to alter viral tropism. Because of the versatility of the Protein A fragment containing viral mosaics, these AAV mosaics offer a general method to target AAV to specific cell types.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, and protocols are cited throughout this application, the disclosures of which are incorporated herein by reference in their entireties for all purposes.

What is claimed is:

1. A DNA library comprising variants of a gene encoding viral capsid or envelope protein, wherein each variant contains a randomly inserted restriction site.

2. The library of claim 1 comprising each possible insertion in a variant gene.

3. The library of claim 1, wherein the DNA library is a plasmid DNA library.

4. The library of claim 1, wherein the gene encodes a parvovirus capsid protein.

5. The library of claim 1, wherein the gene encodes an AAV capsid protein of any AAV serotype.

6. The library of claim 5, wherein the capsid protein is VP1 comprising the amino acid sequence of SEQ ID NO:1.

7. The library of claim 5, wherein the capsid protein is VP2 comprising the amino acid sequence of SEQ ID NO:2.

8. The library of claim 5, wherein the capsid protein is VP3 having the amino acid sequence of SEQ ID NO:3.

9. The library of claim 1, wherein cutting with a restriction enzyme specific for the restriction site produces blunt ends.

10. The library of claim 1, wherein cutting with a restriction enzyme specific for the restriction site produces overhanging ends.

11. The library of claim 10, wherein the restriction site is flanked with sequences encoding a linker.

12. The library of claim 11, wherein the linker comprises a cysteine residue to promote the formation of a disulfide bond between the flanking linkers.

13. A library of virus clones, wherein each clone contains a variant of a gene encoding a viral envelope or capsid protein, wherein each variant contains a randomly inserted restriction site.

14. The library of claim 13 wherein the restriction site is flanked by sequences encoding a linker.

15. The library of claim 14 wherein the linker comprises a cysteine residue to promote the formation of a disulfide bond between the flanking linkers.

16. The library of claim 13 comprising each possible insertion in a variant gene.

17. A library of virus clones, wherein each clone contains a variant of a gene encoding a viral envelope or capsid protein, wherein each variant contains a randomly inserted nucleotide sequence encoding a polypeptide sequence.

18. The library of claim 17, wherein the inserted polypeptide sequence is a targeting sequence.

19. The library of claim 17 comprising each possible insertion in a variant gene.

20. A library of infectious viral particles, wherein each viral particle contains a variant of a gene encoding a viral envelope or capsid protein, wherein each variant contains a randomly inserted polypeptide sequence.

21. The library of claim 20, wherein the inserted polypeptide sequence is a targeting sequence.

22. The library of claim 20, wherein each viral particle further contains at least one other variant capsid or envelope protein or at least one other wildtype capsid or envelope protein.

23. The library of claim 22, wherein the capsid or envelope proteins are from the same virus.

24. The library of claim 22, wherein the capsid or envelope proteins are from at least two different viruses.

25. The library of claim 22, wherein each viral particle contains variant capsid or envelope proteins.

26. The library of claim 22, wherein the targeting polypeptide is a ligand to a receptor expressed by a mammalian cell.

27. The library of claim 22, wherein the viral particle is a parvovirus.

28. The library of claim 27, wherein the viral particle is an AAV of any serotype.

29. A method of preparing a plasmid library comprising a viral gene with a randomly inserted restriction site, which method comprises:
(a) preparing multiple copies of a first plasmid comprising a first selection marker and a viral gene encoding a viral protein;
(b) preparing multiple copies of a second plasmid comprising a second selection marker flanked by transposon sequences, wherein each transposon sequence comprises a restriction site;
(c) preparing a first plasmid library by contacting each copy of the first plasmid with a copy of the second plasmid in the presence of a transposase; and
(d) selecting a first set of plasmids from the first library that comprises both the first and the second selection markers.

30. The method of claim 29 wherein the transposon sequences are Tn7 sequences and the transposase is Tn7-transposase.

31. A method of preparing a library of viral clones, which method comprises transferring each viral gene prepared according to claim 29 into a virus clone, thereby generating a library of viral clones.

32. A method of preparing a library of viral clones comprising a heterologous polypeptide sequence randomly inserted in a viral gene, which method comprises contacting each member of the library of claim 29 with a restriction endonuclease specific for the restriction site, and ligating a nucleotide sequence encoding a heterologous polypeptide sequence into the plasmid at the restriction site.

33. The method according to claim 32 wherein the viral gene is a capsid gene or an envelope gene.

34. A method of preparing a library of pseudotyped viral particles comprising a variant of a capsid gene or envelope gene, which method comprises expressing the library of viral clones of claim 31 in a host cell transfected with a construct that overexpresses a wildtype capsid protein or envelope protein.

35. The method of claim 34, wherein the virus is a parvovirus.

36. The method of claim 35, wherein the parvovirus is an AAV virus of any serotype, the capsid gene is an AAV capsid gene, and the host cell is infected with a helper virus.

37. The method according to claim 36, wherein the host cell is a HEK 293 cell.

38. The method according to claim 36, wherein the helper virus is an adenovirus.

39. The method according to claim 36, wherein the helper virus is a herpes virus.

40. The method according to claim 34, wherein helper functions are provided by a plasmid.

41. The method of claim 36, wherein the AAV capsid gene encodes an AAV VP1 capsid protein comprising the amino acid sequence of SEQ ID NO:1.

42. The method of claim 36, wherein the AAV capsid gene encodes an AAV VP2 capsid protein comprising the amino acid sequence of SEQ ID NO:2.

43. The method of claim 36, wherein the AAV capsid gene encodes an AAV VP3 capsid protein having the amino acid sequence of SEQ ID NO:3.

44. A method of selecting a virus comprising a variant of a first capsid or envelope protein that alters tropism of the virus for a target cell, which method comprises:
(a) infecting host cells with the pseudotyped viral particles of claim 34;
(b) contacting target cells with viral particles produced from the infected cells of step (a) at a multiplicity of infection of less than 1; and
(c) detecting successful infection of the target cells,
wherein successful infection indicates that the tropism of the virus is altered such that it infects the target cell.

45. The method according to claim 44 wherein infection of cells normally infected by the virus is not successful.

46. The method according to claim 44 wherein the host cells in step (a) express a second capsid protein or envelope protein whereby the viral particles contain a mosaic capsid or envelope.

47. The method according to claim 21, wherein the peptide is a member of the group consisting of the HA-epitope, the FLAG-epitope, the serpin-ligand, 4C-RGD, L14, LH, LyP-1, Z34C, VEGF, the c-kit ligand, scFv-ACK2 and scFv-ACK4.

48. A host cell for expressing a recombinant replication-defective virus, which host cell comprises a first construct encoding a first capsid or envelope protein-encoding gene, a second construct encoding a second capsid or envelope protein-encoding gene which is a variant comprising a targeting polypeptide sequence, and a construct comprising a replication-defective recombinant viral genome comprising packaging sequences and a heterologous gene for a protein of interest.

49. The host cell of claim 48, wherein the first capsid or envelope protein-encoding gene is a wildtype gene.

50. The host cell of claim 48, wherein a ratio of the first construct to the second construct is in proportion to a desired ratio of the proteins in a mosaic viral particle.

51. The host cell of claim 48, wherein the virus is a parvovirus and the host cell is infected with a helper virus.

52. The host cell of claim 51, wherein the virus is an AAV of any serotype.

53. The host cell of claim 51, wherein the helper virus is an adenovirus.

54. The host cell of claim 51, wherein the helper virus is a herpes virus.

55. The host cell of claim 48, wherein helper functions are provided by a plasmid.

56. A replication-defective viral vector comprising a capsid or envelope comprising a first capsid or envelope protein, a second capsid or envelope protein that is a variant comprising a targeting polypeptide sequence, and a replication-defective recombinant viral genome comprising packaging sequences and a heterologous gene encoding a protein of interest.

57. A method of producing a mosaic replication-defective viral vector, which method comprises:
(a) co-transfecting a host cell with a first construct comprising a first gene encoding a first capsid or envelope protein, a second construct comprising a second gene encoding a second capsid or envelope protein, which second capsid or envelope protein is a variant comprising a targeting polypeptide sequence, and a third construct comprising a replication-defective recombinant viral genome comprising packaging sequences and a heterologous gene encoding a protein of interest; and
(b) culturing the host cell under conditions that permit generation of recombinant viral particles comprising a mosaic capsid or envelope.

58. The method of claim 57, wherein the first and second constructs are present in a ratio to provide for incorporation of a desired ratio of wild-type to variant capsid or envelope protein in a mosaic replication-defective viral vector produced in the host cell.

59. The method of claim 58, wherein the virus is a parvoviurs.

60. The method of claim 59, wherein the virus is an AAV of any serotype.

61. An AVV mosaic viral vector comprising a gene encoding a protein of interest and further comprising a capsid protein into which the IgG binding domain of Protein A is inserted, wherein said capsid comprises up to about 50% mutant capsid protein.

62. The AAV mosaid viral vector of claim 61, wherein said capsid protein comprises between about 10% and about 25% mutant capsid protein.

63. A method for transducing a cell with a protein of interest comprising contacting said cell with an antibody directed against a surface protein of said cell and the AAV viral vector of claim 61.

64. A method for transducing a cell with a protein of interest comprising contacting said cell with an antibody directed against a surface protein of said cell and the AAV viral vector of claim 62.


SEQUENCE LISTING (18 sequences)

SEQ ID NO: 1 - protein, 735 aa, adeno-associated virus 2 [sequence omitted]

SEQ ID NO: 2 - protein, 598 aa, adeno-associated virus 2
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 533 aa, adeno-associated virus 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 9 aa, artificial
  peptide ligand
YPVDVPDYA

SEQ ID NO: 5 - protein, 9 aa, artificial
  peptide ligand
DYKDDDKYK

SEQ ID NO: 6 - protein, 4 aa, artificial
  peptide ligand
FVLI

SEQ ID NO: 7 - protein, 9 aa, artificial
  peptide ligand
CDCRGDCFC

SEQ ID NO: 8 - protein, 14 aa, artificial
  peptide ligand
QAGTFALRGDNPQG

SEQ ID NO: 9 - protein, 10 aa, artificial
  peptide ligand
HCSTCYYHKS

SEQ ID NO: 10 - protein, 9 aa, artificial
  peptide ligand
CGNKRTRGC

SEQ ID NO: 11 - protein, 4 aa, artificial
  linker peptide
  MISC_FEATURE (1)..(3): residues 1-3 can be included 0-3 times
GGSC

SEQ ID NO: 12 - protein, 4 aa, artificial
  linker peptide
  MISC_FEATURE (2)..(4): residues 2-4 can be included 0-3 times
CGGS

SEQ ID NO: 13 - protein, 4 aa, artificial
  linker peptide
  MISC_FEATURE (1)..(3): residues 1-3 can be included 0-3 times
ALSC

SEQ ID NO: 14 - protein, 4 aa, artificial
  linker peptide
  MISC_FEATURE (2)..(4): residues 2-4 can be included 0-3 times
CALS

SEQ ID NO: 15 - protein, 5 aa, artificial
  peptide insert
PCLNS

SEQ ID NO: 16 - protein, 5 aa, artificial
  peptide insert
GCLNT

SEQ ID NO: 17 - protein, 5 aa, artificial
  peptide insert
LFKHN

SEQ ID NO: 18 - protein, 34 aa, s. Aureus
FNMQCQRRFYEALHDPNLNEEQRNAKIKSIRDDC